(19) United States
(12) Patent Application Publication
(10) Pub. No.: US 2022/0051754 A1
(43) Pub. Date: Feb. 17, 2022

(54) DNA ANALYZER WITH SYNTHETIC ALLELIC LADDER LIBRARY

(71) Applicant: Life Technologies Corporation, Carlsbad, CA (US)

(72) Inventor: Mattias Vangbo, Fremont, CA (US)

(73) Assignee: Life Technologies Corporation, Carlsbad, CA (US)

(21) Appl. No.: 17/402,400

(22) Filed: Aug. 13, 2021

Related U.S. Application Data
(60) Provisional application No. 63/067,289, filed on Aug. 18, 2020.
(60) Provisional application No. 63/066,218, filed on Aug. 15, 2020.

(51) Int. Cl. G16B 30/10; G16B 35/20; C12N 15/10; B01L 7/00; G01N 27/447

(52) U.S. Cl. CPC G16B 30/10; G16B 35/20; C12N 15/1089; B01L 2300/18; G01N 27/44791; G01N 27/44721; B01L 7/52

(57) ABSTRACT

A method of testing a biological sample comprising deoxyribonucleic acid (DNA) molecules for presence of a plurality of alleles is described, wherein DNA fragments obtained using the biological sample and corresponding to different alleles have different fragment sizes. A capillary electrophoresis (CE) instrument is used to obtain test fragment sizing data for the biological sample. A pre-computed model is used to dynamically determine one or more synthetic allelic ladders, where the pre-computed model is derived via analysis of a plurality of fragment sizing data sets obtained from a plurality of previous allelic ladder sample runs conducted using CE instruments. The one or more synthetic or experimentally derived allelic ladders are used to find a sufficient fit to the test fragment sizing data to identify which of the plurality of alleles are present in the biological sample. The statistical analysis may comprise a principal component analysis including two principal components.

### BACKGROUND

The present disclosure relates generally to systems, devices, and methods for deoxyribonucleic acid (DNA) analysis, and more specifically to systems, devices, and methods for DNA fragment analysis of short tandem repeat (STR) sequences for forensic or paternity testing purposes using capillary electrophoresis.

Since it has been estimated that over 99.7% of the human genome is the same from individual to individual, regions that differ need to be found in the remaining 0.3% in order to tell people apart at the genetic level. There are many repeated DNA sequences scattered throughout the human genome.

Eukaryotic genomes are full of repeated DNA sequences (Ellegren 2004). These repeated DNA sequences come in all sizes and are typically designated by the length of the core repeat unit and the number of contiguous repeat units or the overall length of the repeat region. Long repeat units may contain several hundred to several thousand bases in the core repeat.

DNA regions with repeat units that are 2 base pairs (bp) to 7 bp in length are called microsatellites, simple sequence repeats (SSRs), or most usually short tandem repeats (STRs). STRs have become popular DNA repeat markers because they are easily amplified by polymerase chain reaction (PCR) without the problems of differential amplification. This is because both alleles from a heterozygous individual are similar in size since the repeat size is small. The number of repeats in STR markers can be highly variable among individuals, which makes these STRs effective for human identification purposes.

Historically, DNA sequencing products were separated using polyacrylamide gels that were manually poured between two glass plates. Capillary electrophoresis using a denaturing flowable sieving polymer (also referred to herein as a “gel”) has largely replaced the use of older gel separation techniques due to significant gains in workflow, throughput, and ease of use. Fluorescently labeled DNA fragments are separated according to molecular weight. Because there is no need to pour gels with capillary electrophoresis, DNA sequence analysis using CE is automated more easily and can process more samples at once.

An STR typing kit consists of five components: a PCR primer mixture containing oligonucleotides designed to amplify a set of STR loci, a PCR buffer containing deoxynucleotide triphosphates, MgCl₂, and other reagents necessary to perform PCR, a DNA polymerase, which is sometimes premixed with the PCR buffer, an allelic ladder sample with common alleles for the STR loci being amplified to enable calibration of allele repeat size, and a positive control DNA sample to verify that the kit reagents are working properly. (See John M. Butler, Chapter 5 in Advanced Topics in Forensic DNA Typing: Methodology, 2012, p. 99-139). To enable comparison between samples, an internal size standard, also called internal lane standard (ILS), is also added to each test sample and allelic ladder sample.

During capillary electrophoresis, the extension products of the cycle sequencing reaction enter the capillary as a result of electrokinetic injection. A voltage applied to the buffered sequencing reaction forces the negatively charged fragments into the capillaries, where the voltage is applied across the gel, and a thus a portion of the voltage is applied over the fragments. The extension products are separated by size based on their conformation and total charge. The electrophoretic mobility of the sample can be affected by the run conditions: the buffer type, concentration, and pH, the run temperature, the amount of voltage applied, and the type of polymer used.

Shortly before reaching the positive electrode, the fluorescently labeled DNA fragments, separated by size, move across the path of a laser beam. The laser beam causes the dyes on the fragments to fluoresce, and the fluorescence is detected by an optical detector. Data collection software converts the detected fluorescent signal to digital data, then records the data, for example, in a comma separated text file. Because each dye emits light at a different wavelength when excited by the laser, several sets of fragments of similar size can be detected and distinguished in one capillary injection.

In capillary electrophoresis (CE), a biological sample, such as a nucleic acid sample, is injected at the inlet end of the capillary, into a denaturing separation medium (sometimes referred to by those skilled in the art as a “gel”) in the capillary, and an electric field is applied to the capillary ends. The different nucleic acid components in a sample, e.g., a polymerase chain reaction (PCR) mixture or other sample, migrate to the detector point with different velocities due to differences in their electrophoretic properties. Consequently, they reach the light detector (usually a fluorescence detector operating in the visible light range or an ultraviolet (UV) absorbance detector) at different times. Results present as a series of detected peaks, where each peak represents ideally one nucleic acid component or species of the sample.

The magnitude of any given peak, including an artifact peak, is most often determined optically on the basis of either UV absorption by nucleic acids, e.g., DNA, or by fluorescence emission from one or more labelled dyes associated with the nucleic acid. UV and fluorescence detectors applicable to nucleic acid CE detection are well known in the art.

CE capillaries themselves are frequently quartz, although other materials known to those of skill in the art can be used. There are a number of CE systems available commercially, having both single and multiple-capillary capabilities. The methods described herein are applicable to any device or system for CE of nucleic acid samples.

### SUMMARY

In DNA fragment analysis, STR fragments of unknown identity are compared to a set of fragments of known sizes, also known as the internal lane standard (ILS). By means of interpolation, an apparent size of the unknown fragments can be determined, and the identity of the fragment can be inferred. One complication, however, well known among those skilled in the art, is that said apparent size will vary from time to time due to temperature effects, and the type and condition of the gel, among other factors. The size that is measured for a given STR fragment in DNA fragment analysis is not its “true” size, it only means that at that particular time, under those particular conditions, the STR fragment migrated at the same speed a hypothetical ILS fragment of that same size would.

As a simple example, temperature is found by experiment to strongly affect migration, and hence the size that is measured for a molecule. Overall, warmer temperatures will mean faster migration, but as long as the sample and ILS migration rates change in unison, this will not affect sizing. However, usually there is a small difference in the change of rates for the different fragments, and commonly the sample fragments will lag the increased migration rate of the ILS fragments and will therefore get sized larger at higher temperatures. On the other hand, some sample fragments may instead migrate faster relative to the ILS and therefore get sized smaller. This will depend on the specific fragments and the selection of ILS fragments. Any difference in the change of migration rate between and allele and the ILS will cause the sizing of the peak to change. For example, at a control temperature of 60 degrees Celsius, versus a control temperature of 50 degrees Celsius, a given DNA fragment can be assigned a size that is 1 base pair larger or more.

On a CE instrument that can run a set of samples in parallel, these variations can mostly be accommodated for by including a reference sample with each set. A reference sample, for STR analysis purposes also known as an allelic ladder, is a sample where most or all possible fragments for each allele to be investigated have been assembled into a single sample. As the set is known, the identity of each fragment can be determined and associated with an apparent size, as it is compared with the ILS, under the given conditions.

For a single capillary instrument, such as the RapidHIT™ ID System manufactured by Applied Biosystems, Inc., the reference sample cannot be performed simultaneously with the samples, but instead it is common to perform the reference run under as similar conditions as possible as the sample run, and within a short period of time. This can be disadvantageous in forensic analysis, where crime scene investigations and accident scene investigations often demand fast turnaround times for human identification and DNA testing of numerous DNA samples.

Many times, a system will, as a back-up, have a library of older allelic ladders to compare with and the system has an algorithm to make a selection to find a sufficient fit or best fit known allelic ladder that can be used to identify the alleles in the test sample. As discussed above, systematic variations in temperature, gel degradation, buffers, voltage changes, and gel lot, may occur from run-to-run and affect fragment sizing data measurements. Noise effects from current, optical noise, gel inhomogeneity, impurities, and secondary structure may also occur.

In addition, these libraries of older allelic ladders may not be fully representative of typical or valid operating ranges of the CE instruments and reliance on these libraries could potentially impact the accuracy of the DNA identification process. One issue in libraries of older allelic ladders arises in how they are assembled (e.g., manually selected) and how well does the library cover the variations. The density and dimensionality of the library's coverage, as well as how representative the included ladders are, may also have an impact. Even if all external parameters can be held constant in theory, differences in composition, injection and noise in the measurements can affect how well it represents or fits a typical or particular sample. Another issue in using older allelic libraries is how to select the best fit or sufficiently fit allelic ladder from the allelic ladder library. If the ladders in the ladder library have significant noise or other effects that deviate from a typical or particular sample run, the risk of ambiguous selection increases. For example, ambiguity in ladder selection can occur if two ladders in the ladder library are very similar. In some cases, the peaks in a test sample may be identified identically regardless of which of two ladders is selected for the identification, and the ambiguity is of no concern. In another case, two very different ladders can provide a sufficient fit to the test sample, and only small differences, such as noise, may determine which ladder is ultimately selected as reference for the sample. This has a higher risk of happening if the test sample includes none or a very small numbers of peaks, for example less than five or ten.

An incorrect identification of a DNA fragment in forensic analysis can have very severe implications, e.g. in criminal investigations by law enforcement, and in judicial criminal and civil trials where the fates of lives of individuals are decided. Therefore, methods to improve the accuracy and speed up the analysis time of sample identification using DNA fragment analysis are needed.

Embodiments of the present invention describe a method of testing a biological sample comprising deoxyribonucleic acid (DNA) molecules for presence of a plurality of alleles, wherein DNA fragments obtained using the biological sample and corresponding to different alleles have different fragment sizes. A capillary electrophoresis (CE) instrument is used to obtain test fragment sizing data for the biological sample. A pre-computed model is used to generate one or more synthetic or experimentally derived allelic ladders, where the pre-computed model is derived via statistical analysis of a plurality of fragment sizing data sets obtained from a plurality of previous allelic ladder sample runs conducted using CE instruments. The one or more synthetic allelic ladders are used to find a sufficient fit to the test fragment sizing data to identify which of the plurality of alleles are present in the biological sample. The statistical analysis may comprise a principal component analysis (PCA) including two principal components.

A statistical model incorporating PCA and incorporating two principal components leverages the notion that for an otherwise fixed and stable DNA fragment analysis system, particularly those incorporating CE instruments, two of the most significant effects affecting the apparent size of a DNA fragment are temperature and to what extent the gel has degraded.

In one embodiment a pre-computed model can be developed by measuring the response of each DNA fragment from each of these effects (temperature and gel degradation) experimentally, In particular, the response of each DNA fragment being analyzed can be determined from experiments where the temperature and gel degradation are tightly controlled to derive an empirical migration model. By linearly combining these responses using a linear regression analysis, the apparent size of a fragment at any set of conditions can be estimated. It can be empirically shown that such estimations will be accurate for limited range of conditions.

A different approach to determine these responses of the DNA fragments to gel degradation and temperature effects is to assemble the apparent sizes from many sample runs where the temperature (e.g., room temperature and/or separation heater temperature) and gel degradation have varied at random and/or are unknown, and develop a pre-computed model by performing a principal component analysis (PCA). This approach has the additional benefit of reducing noise since such an analysis generally will take many more runs into account. A PCA analysis, however, will not provide the response of temperature and gel degradation separately; rather, it will provide two set of responses that can be linearly combined to make the same set of estimations as the measurement of the various controlled isolated temperature and degradation responses as described above. In particular, the responses from primarily or largely isolated effects of temperature and gel degradation respectively may be reconstructed as a linear combination of the PCA output. The PCA analysis will also indicate if there are additional parameters that need to be considered.

Regardless of the approach taken to build the pre-computed model, such a model is able to predict the apparent size of any fragment at any condition for which the model is valid. Hence it is possible to predict the outcome of a reference run under any set of conditions, and by reverse comparison, it is possible to infer under what conditions any reference run or any sample run was made.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1 illustrates a capillary electrophoresis-based DNA analysis system in accordance with an embodiment of the present invention;

FIG. 2A illustrates an exemplary DNA analysis instrument in accordance with an embodiment of the present invention;

FIG. 2B illustrates two perspective views of an exemplary sample cartridge for the system of FIG. 2A that may be used in accordance with an embodiment of the present invention;

FIG. 2C illustrates a perspective view of an exemplary primary cartridge for the system of FIG. 2A that may be used in accordance with an embodiment of the present invention;

FIG. 3 illustrates a workflow process for a CE-based DNA analysis system in accordance with an embodiment of the present invention;

FIG. 4 illustrates an exemplary set of scans from an STR analysis sample run that may be displayed in accordance with an embodiment of the invention;

FIG. 5 illustrates a prior art STR analysis workflow process that may be used in accordance with an embodiment of the invention;

FIG. 6 illustrates a STR analysis workflow process in accordance with an embodiment of the present invention;

FIG. 7 illustrates a process for building an empirical migration model in accordance with an embodiment of the present invention;

FIG. 8A illustrates experimental results for a gel degradation variable for an empirical migration model in accordance with an embodiment of the present invention;

FIG. 8B illustrates experimental results for a temperature variable for an empirical migration model in accordance with an embodiment of the present invention;

FIG. 9 illustrates a process for building a migration model based on principal component analysis (PCA) in accordance with an embodiment of the present invention;

FIG. 10 illustrates a graphical representation of principal components generated in a PCA-based migration model in accordance with an embodiment of the present invention;

FIG. 11 illustrates a PCA-based STR analysis workflow process in accordance with an embodiment of the present invention;

FIG. 12 illustrates a PCA-based STR analysis workflow process in accordance with another embodiment of the present invention;

FIG. 13A illustrates a graphical representation of a PCA analysis of a manually aggregated ladder library;

FIG. 13B illustrates a graphical representation of a PCA analysis of a synthetic ladder library in accordance with an embodiment of the present invention;

FIG. 14 illustrates a PCA-based process for generating a synthetic allelic ladder in accordance with an embodiment of the present invention;

FIG. 15 illustrates an exemplary PCA-based migration model in accordance with an embodiment of the present invention;

FIG. 16 illustrates a PCA-based CE instrument validation process using synthetic allelic ladders in accordance with an embodiment of the present invention;

FIG. 17 illustrates a block diagram of an exemplary computing device that may incorporate embodiments of the present invention.

While the invention is described with reference to the above drawings, the drawings are intended to be illustrative, and other embodiments are consistent with the spirit, and within the scope, of the invention.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

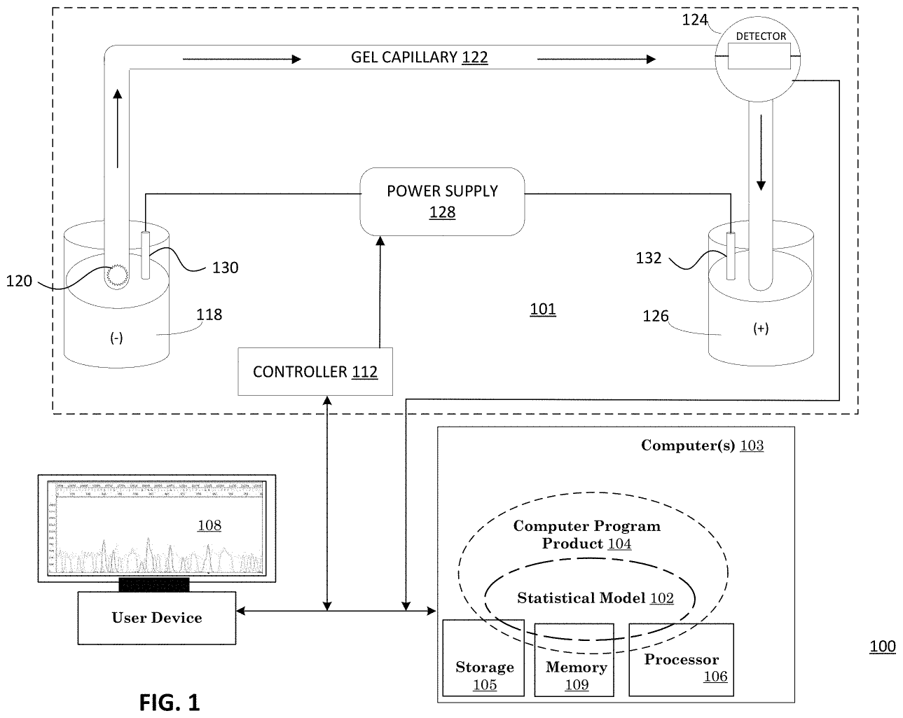

FIG. 1 illustrates System 100 in accordance with an exemplary embodiment of the present invention. System 100 comprises capillary electrophoresis (“CE”) DNA analysis instrument 101, one or more computers 103, and user device 107.

In one embodiment of the present invention, system 100 comprises an exemplary commercial CE device as defined in this specification that may include the Applied Biosystems, Inc. RapidHIT™ ID System and/or RapidHIT™ 200 System. However, other exemplary commercial CE devices that may be used in embodiments of the present invention include, but are not limited to the following: Applied Biosystems, Inc. (ABI) genetic analyzer models 310 (single capillary), 3130 (4 capillary), 3130xL (16 capillary), 3500 (8 capillary), 3500xL (24 capillary), and the SeqStudio genetic analyzer models; DNA analyzer models 3730 (48 capillary), and 3730xL (96 capillary); as well as the Agilent 7100 device, Prince Technologies, Inc.'s PrinCE™ Capillary Electrophoresis System, Lumex, Inc.'s Capel-105™ CE system, and Beckman Coulter's P/ACE™ MDQ systems, among others. Embodiments of the present invention may also be contemplated for use in other electrophoresis systems, such as gel electrophoresis, that generate DNA fragment sizing data.

Referencing system 100 in FIG. 1, a CE DNA analysis instrument 101 in one embodiment comprises a source buffer 118 containing buffer and receiving a fluorescently labeled sample 120, a gel capillary 122, a destination buffer 126, a power supply 128, and a controller 112. The source buffer 118 is in fluid communication with the destination buffer 126 by way of the capillary 122. The power supply 128 applies voltage to the source buffer 118 and the destination buffer 126 generating a voltage bias through a cathode 130 in the source buffer 118 and an anode 132 in the destination buffer 126. The voltage applied by the power supply 128 is configured by a controller 112 operated by the computing device 103. Fluorescently labeled sample 120 at the source buffer 118 is pulled through the capillary 122 by the voltage gradient, and optically labeled nucleotides of the DNA fragments within the sample are detected as they pass through an optical detector 124 on the way to destination buffer 126. Differently sized DNA fragments within the fluorescently labeled sample 120 are pulled through the capillary at different times due to their size.

The optical sensor 124 detects the fluorescent labels on the nucleotides as an image signal and communicates the image signal to the computing device 103. The computing device 103 aggregates the image signal as sample data and utilizes a computer program product 104 to operate a statistical model 102 to transform the sample data into processed data, including one or more basecall sequences and/or fragment sizes, and generate a DNA profile, including, e.g., one or more electropherograms that may be shown on a display 108 of user device 107. In one embodiment of the invention, DNA analysis instrument 101 may comprise one or more versions of the Applied Biosystems RapidHIT™ ID System or RapidHIT™ 200 System.

Instructions for implementing pre-computed statistical model 102 reside on computing device 103 in computer program product 104 which is stored in storage 105 and those instructions are executable by processor 106. In one embodiment of the invention, computer program product 104 may comprise one or more versions of the Applied Biosystems RapidLINK™ Software product, which may be accessed by computing device 103 in whole or in part from a remote location through a network interface. When processor 106 is executing the instructions of computer program product 104, the instructions, or a portion thereof, are typically loaded into working memory 109 from which the instructions are readily accessed by processor 106. In one embodiment, computer program product 104 is stored in storage 105 or another non-transitory computer readable medium (which may include being distributed across media on different devices and different locations). In alternative embodiments, the storage medium is transitory.

In one embodiment, processor 106 may comprise multiple processors which may comprise additional working memories (additional processors and memories not individually illustrated) including a graphics processing unit (GPU) comprising at least thousands of arithmetic logic units supporting parallel computations on a large scale. GPUs are often utilized in machine learning applications because they can perform the relevant processing tasks more efficiently than can typical general-purpose processors (CPUs). Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application-specific), field programmable gate arrays and the like, or combinations thereof. In some embodiments, however, a processor such as processor 106 may be implemented as one or more general purpose processors (preferably having multiple cores) without necessarily departing from the spirit and scope of the present invention.

User device 107 incudes a display 108 for displaying results of processing carried out by statistical model 102. In alternative embodiments, statistical model 102, or a portion of it, may be stored in storage devices and executed by one or more processors residing on CE instrument 101 and/or user device 107. Such alternatives do not depart from the scope of the invention.

As discussed above, DNA profiling from samples recovered at crime scenes has become a “gold standard” of forensic testing. Processing forensic evidence from crime scenes involves numerous labor intensive-steps: sample selection, DNA extraction and quantification, PCR amplification of short tandem repeats (STR) and generation of the DNA profile by capillary electrophoresis (CE). For urgent samples, time-to-result is often far longer than desired by today's law enforcement demands.

Rapid DNA systems are highly automated sample-to-answer platforms for generating DNA profiles. An exemplary Rapid DNA system used in embodiments of the present invention is the Applied Biosystems RapidHIT™ ID System, optimized for decentralized operation for use in both crime laboratories and by unskilled users in law enforcement offices or other non-laboratory settings. Further information on the RapidHIT™ ID System is available in the Applied Biosystems RapidHIT™ ID System v1.0 User Guide (Pub. No. MAN0018039), which is hereby incorporated by reference in its entirety. Another exemplary Rapid DNA system used in some embodiments of the present invention is the Applied Biosystems RapidHIT™ 200 System.

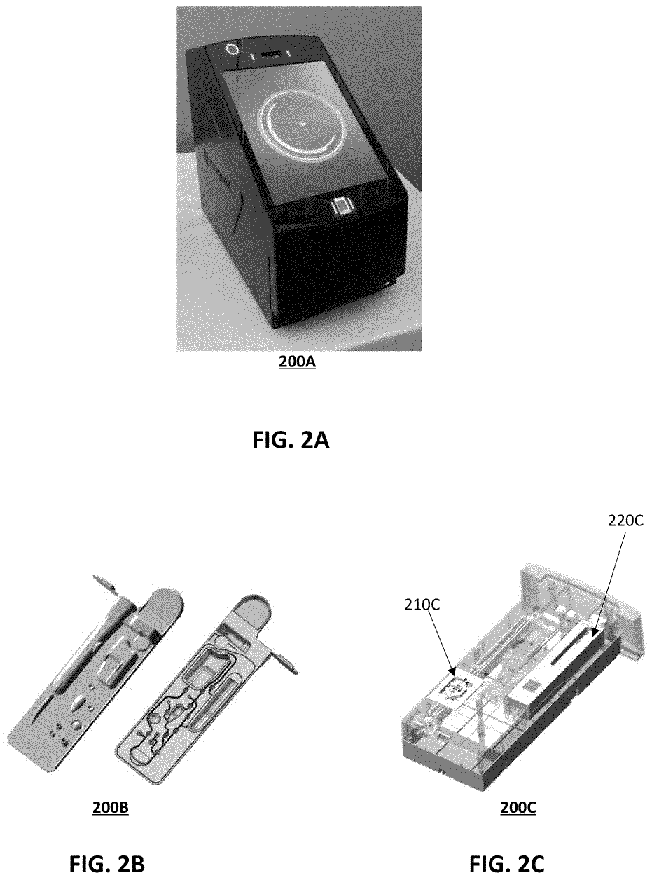

An exemplary DNA analysis instrument 200A used in some embodiments of the present invention is shown in FIG. 2A. An exemplary embodiment of system 200A comprises the Applied Biosystems RapidHIT™ ID System, although other embodiments of system 200A may comprise the Applied Biosystems RapidHIT™ 200 System. In this embodiment, instrument 200A comprises a fully automated, sample-to-CODIS (Combined DNA Index System) system for STR-based human identification (HID) that may process presumed single-source samples in less than 90 minutes with less than one minute of hands-on time. Instrument 200A may perform some analysis using a library of one or more allelic ladders provided on the instrument 200A. After performing capillary electrophoresis and generating an STR profile, system 200A transfers the generated fragment sizing data set to RapidLINK™ software for processing, and if necessary, manual profile review. RapidLINK™ also manages reagent supplies and operator access across a network of DNA devices. In one embodiment of the invention, RapidLINK™ software may reside on computer(s) 103 as computer program product 104 and contain instructions for performing further analysis. Further information on RapidLINK™ software is available in the Applied Biosystems RapidLINK™ Software v1.0 User Guide (Pub. No. MAN0018038), which is hereby incorporated by reference in its entirety.

In one embodiment of the present invention, system 200A is designed to use one or more sample cartridges for processing DNA samples. Such sample cartridges may process DNA samples from crime scenes, or DNA samples on buccal swabs (where, e.g., the inside of a person's cheek is swabbed for DNA). One exemplary cartridge used in embodiments of the present invention is the RapidHIT™ ACE sample cartridge 200B for processing buccal swabs, shown in FIG. 2B. In one embodiment, cartridge 200B utilizesGlobalFiler® Express or AmpFLSTR® NGM SElect™ Express (Thermo Fisher Scientific, Inc.) multiplexes. PCR amplification, electrophoresis, and analysis of the amplified products are all done within system 200A.

Aside from sample cartridges such as exemplary sample cartridge 200B, other consumables for instrument 200A, including capillary 210C and a gel cartridge 220C, are provided on primary cartridge 200C shown in FIG. 2C, which is installed on instrument 200A and may be replaced periodically as part of regular maintenance of instrument 200A. Instrument 200A also includes an internal environmental sensor that monitors temperature and humidity.

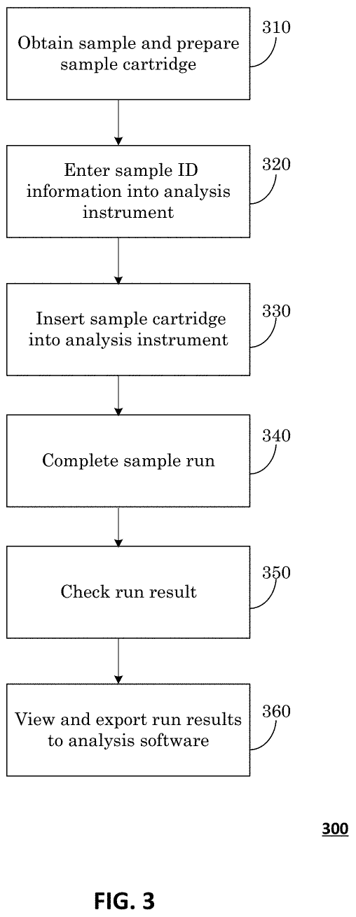

FIG. 3 comprises a STR analysis workflow 300 used in an embodiment of the present invention. In one embodiment of the present invention, system 100 uses several components, including instrument 200A, sample cartridge 200B and computer program product 104. In step 310, a sample is obtained (e.g., from a buccal swab) and a sample cartridge 200B containing STR chemistry is prepared. Next, a user interface on instrument 200A will upon activation/invocation, guide the user through routine use, including entering the sample ID into the instrument 200A in step 320 and inserting the sample cartridge into instrument 200A in step 330 to begin the sample run. In step 340, instrument 200A will generate a DNA profile in approximately 90-110 minutes. When the sample run is completed in step 350, the sample cartridge should be removed from instrument 200A, and instrument 200A will display a result screen. Exemplary status indicators for instrument 200A include: Green, showing that a DNA profile was generated and does not contain quality score flags, Yellow, showing that a DNA profile was generated with one or more quality score flags, or Red, signifying that a DNA profile was not generated. In step 360, generated DNA profiles may be exported to computer 103 for further analysis in computer program product 104.

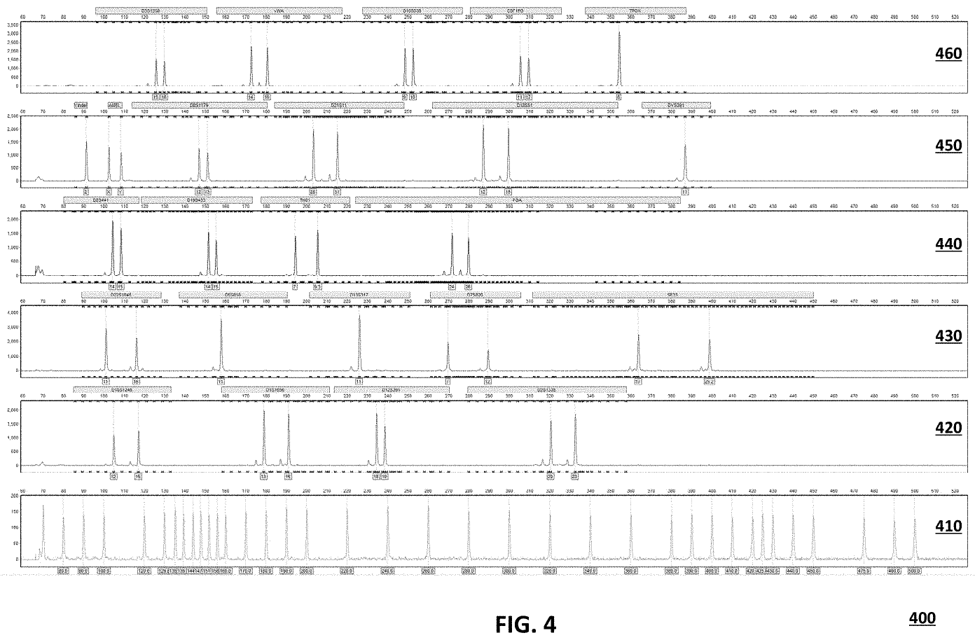

FIG. 4 illustrates an exemplary set of scans from an STR analysis sample run in accordance with an embodiment of the invention. This set of scans comprises a DNA profile generated by instrument 200A. For each scan, the horizontal x-axis running along the top of each scan shows the number of base pairs, and the peaks going up along the y-axis show the fluorescence values where the fluorescently labelled fragment is detected.

Scan 410 represents an internal lane standard (ILS), which comprises a set of DNA fragments of known sizes. The boxes below each peak, along the x-axis at the bottom of scan 410 show the number of base pairs for a fragment detected at that peak. Scans 420-460 represent 5 different fluorescent dye markers (e.g., FAM, VIC, NED, TAZ, SID) shown in different colors used to label alleles at various DNA loci. The rectangular boxes running along the top of each of scans 420-460 are labeled with the name of a DNA locus and show the size range of the alleles for that locus, and the numbered boxes running along the bottom x-axis of each of scans 420-460 show the peak where the allele was detected, and is labeled with the allele size. Each sample generally shows 2 peaks (representing different alleles) for each DNA locus representing chromosomal DNA from the mother and from the father, although some loci may only have one peak. An allelic ladder therefore represents a set of known alleles for each of a plurality of DNA loci. However, as discussed elsewhere in this specification, STR analysis sample run fragment sizing results for test samples and allelic ladders can vary from day to day or time to time, but not necessarily at random. Temperature variations, gel age, gel type, and gel condition, among other factors, can all cause apparent fragment size to vary. One way to accommodate these variations is to include a reference sample, such as an allelic ladder sample, with each set of test samples run.

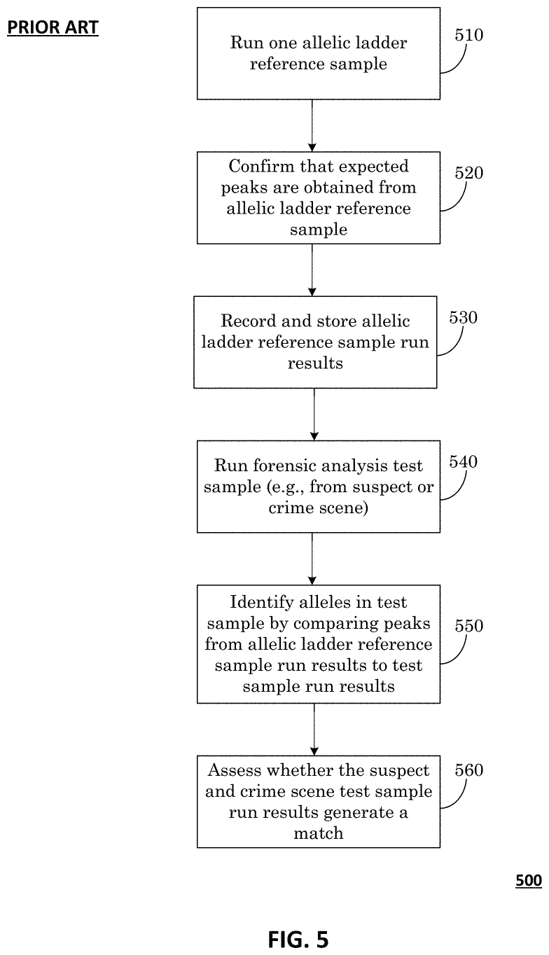

FIG. 5 illustrates a prior art STR analysis workflow process that may also be used in embodiments of the present invention. In step 510, an allelic ladder reference sample run is performed. On an instrument that can run a set of samples in parallel, the variations discussed above can be accommodated for by including a reference sample with each set. On a single capillary instrument, such as the RapidHIT™ ID instrument, it is common to perform the reference sample run preferably within as similar conditions as possible as the test sample, and within a short period of time on the same instrument. In step 520, the user confirms that the expected peaks are obtained from the allelic ladder reference sample. In step 530, the allelic ladder reference sample run results are recorded and stored for further analysis. In step 540, one or more test samples from a subject (e.g., a forensic sample obtained from a suspect, a person of interest, or a crime scene) are run on the instrument. In step 550, the alleles in the test sample are identified by comparing the peaks from the allelic reference sample run results to the test sample run results. In step 560, it is then determined whether the test sample of the subject matches that of a reference (e.g., matches the identity of an individual contained in a criminal database, or of a suspect or victim).

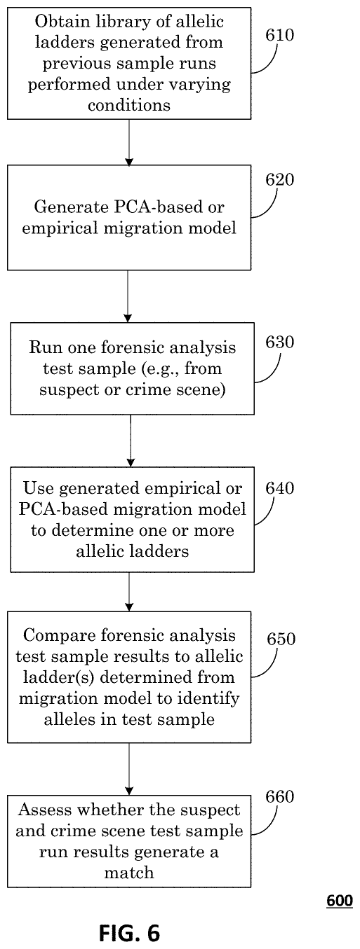

FIG. 6 illustrates an STR analysis workflow process 600 in accordance with an embodiment of the present invention that may obviate the need for a reference sample run as used in known approaches such as those described in FIG. 5 above, and thereby make the DNA analysis and identification process faster and/or more accurate. The approach of FIG. 6 makes use of the observation that for an otherwise fixed and stable system, two of the most significant effects affecting the apparent size of a fragment in a sample run on a CE instrument are temperature and to what extent the gel has degraded. One reason why temperature and gel degradation have a significant effect on perturbations in apparent fragment sizes for a given allele is that these two variables are virtually impossible to hold constant.

In step 610, the process starts by assembling the apparent sizes from many sample runs where the temperature and gel degradation (and possibly additional parameters, such as instrument or sample cartridge type/model) have varied. In one approach in step 620, an empirical model may be constructed to determine the response of each fragment to each of these effects (e.g., temperature and gel degradation) by performing a series of experiments where a series of calibration runs are performed on allelic ladder samples, and where the temperature and gel degradation are tightly controlled. By linearly combining these responses, the apparent size of a fragment at any set of conditions can be estimated. It can also be shown via experiment and empirical observation that such estimations will be accurate within a limited range of the each of the above conditions.

Alternatively, in step 620, a different approach to take into account these effects on fragment sizing data is to assemble the apparent fragment sizes for each allele from a training set of many previous sample runs where the temperature and gel degradation have varied at random (and/or are unknown) across a diverse set of use cases, and perform a principal component analysis (PCA) to generate a PCA-based migration model. This PCA-based approach has the additional benefit of reducing noise since this type of statistical analysis can and/or will generally take many more runs into account than the above-described empirical approach. As may be understood by those skilled in the art, a PCA-based analysis will not provide the response of temperature and gel degradation separately; rather, it will provide two sets of responses that can be linearly combined to make the same set of estimations as the isolated temperature and gel degradation responses derived by controlled experiments in the empirical migration model as discussed above. In particular, it is expected that the responses from the isolated effects of temperature and gel degradation respectively can be reconstructed as a linear combination of the PCA output. As noted elsewhere in this text, PCA should be considered as representative of a number of “correlation-finding” or dimensionality reduction analysis methods known in the art. It should also be noted that such analysis methods may utilize two or more parameters to sufficiently capture the variations in allelic ladders due to variations in migration behavior.

Regardless of the approach taken to build the model, such a model is able to predict the apparent size of any fragment at any condition for which the model is valid. Hence, it is possible to predict the outcome of a reference run under any set of conditions, and by reverse comparison, it is possible to infer under what conditions a reference run was made.

Thus, regardless of whether a PCA-based or empirical migration model is selected, accurate analysis may be accomplished without the need for a separate reference sample run to be completed in parallel or within a short time period and under the same or similar conditions as the test sample run. In step 630, a test biological sample (e.g., from a client, subject, suspect, victim, or crime scene) is run for DNA forensic or paternal analysis. In step 640, the generated empirical or PCA-based migration model is used to determine one or more allelic ladders that are sufficiently fit to the test sample. In step 650, the forensic analysis test sample results are compared to the allelic ladder(s) determined in the migration model to identify the alleles in the test sample. The process concludes in step 660 after all test sample runs have been completed, and it can be determined whether the suspect, victim and/or crime scene test sample run results generate a match.

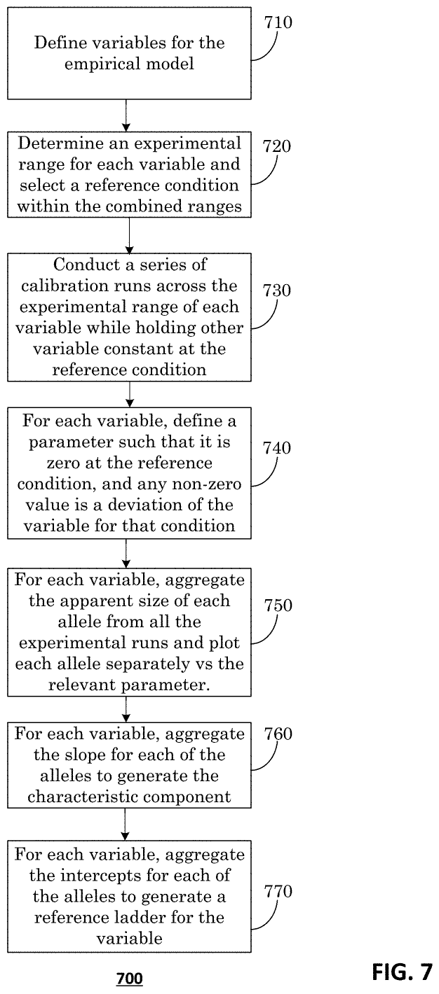

FIG. 7 illustrates a process for building an empirical migration model in accordance with an embodiment of the present invention. In step 710, gel degradation and temperature are defined as the two variables for the empirical model. In other embodiments of the invention, other CE systems may utilize two or more variables or parameters to cover all variations among allelic ladders. An experimental range for each variable is determined and a reference condition within the experimental ranges for each variable is selected in step 720.

In step 730, an experiment is conducted where for each variable, an experiment is conducted where a series of calibration runs on allelic ladder samples are performed across the relevant range of the variable while holding the other variable constant at the reference condition.

In one embodiment of the present invention, the reference condition can be used as one of the data points in each experiment where the experimental conditions are common in both experiments, and one variable may be held fixed at the reference condition while the other variable is varied. Regardless of whether the reference condition is explicitly included in the experiments or not, in one embodiment of the invention the reference condition is strategically selected, e.g., at the center of the combined range.

In step 740, a parameter is defined for each variable such that it is zero at the reference condition, and that any non-zero value indicates a deviation of the variable for that condition. The parameter does not have to be a linear function of the variable. For example, selecting log(T)-log(T₀) as the parameter, where T is the temperature and T₀ is the temperature of the reference condition, is valid should it be found to improve the accuracy of the final model. In one embodiment of the present invention, gel conductivity or time of degradation at a fixed temperature is used as a parameter (or proxy) for gel degradation.

In step 750, for each variable, the apparent sizes for each allele as measured in the experimental runs are aggregated and each allele is plotted separately versus the parameter being studied. Next, the regression parameters (linear fit parameters) are determined for each plot (each allele). In step 760, for each variable, the slope of each of the alleles is aggregated. This set constitutes the “characteristic component” for this variable.

In step 770, for each variable, the intercepts for each of the alleles is aggregated. This set constitutes a “reference ladder” for the variable. If the empirical model experiments are carried out with fidelity in a controlled and rigorous manner as discussed, the reference ladders for the two variables should be very similar, and very similar to the result(s) from the experimental ladders at the reference condition. In one embodiment of the present invention, one can by discretion select a common reference ladder by taking the average of the reference ladders for each of the alleles, or the average of several experimental ladders at the reference condition, whichever proves to yield the better accuracy of the empirical model (when compared to the combined data set from the experiment or a set of verification data).

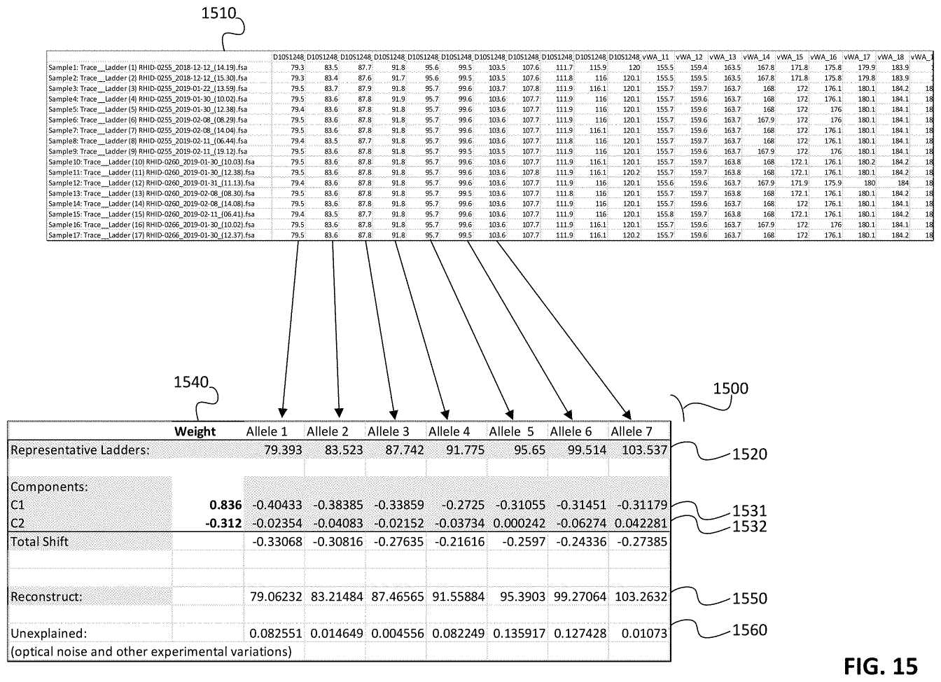

A model generated using the empirical linear regression method of FIG. 7 can be of similar form to the PCA-generated model illustrated and discussed further below in the context of FIG. 15. In other words, the model will include components corresponding to, for example, temperature and gel age, but those components can be expressed without reference to any particular physical parameters, with each component having given normalized values for each allele. An additional “weight” value for each component is added to the model to allow different ladders to be generated from the model until a sufficiently good fitting ladder is found. This is shown and discussed further in the context of FIG. 15. For convenience, in one embodiment of the present invention, the value of each component may be normalized such that its largest absolute value is equal to one, such that the unit of the corresponding weight is in base pairs. Such normalized values are included in this specification for ease of discussion, but are not required.

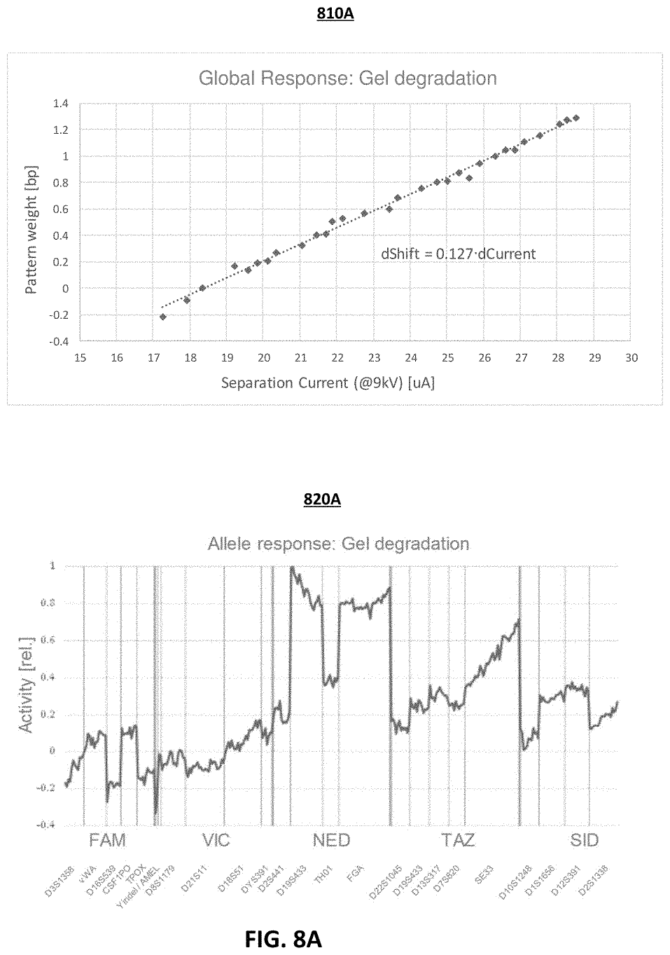

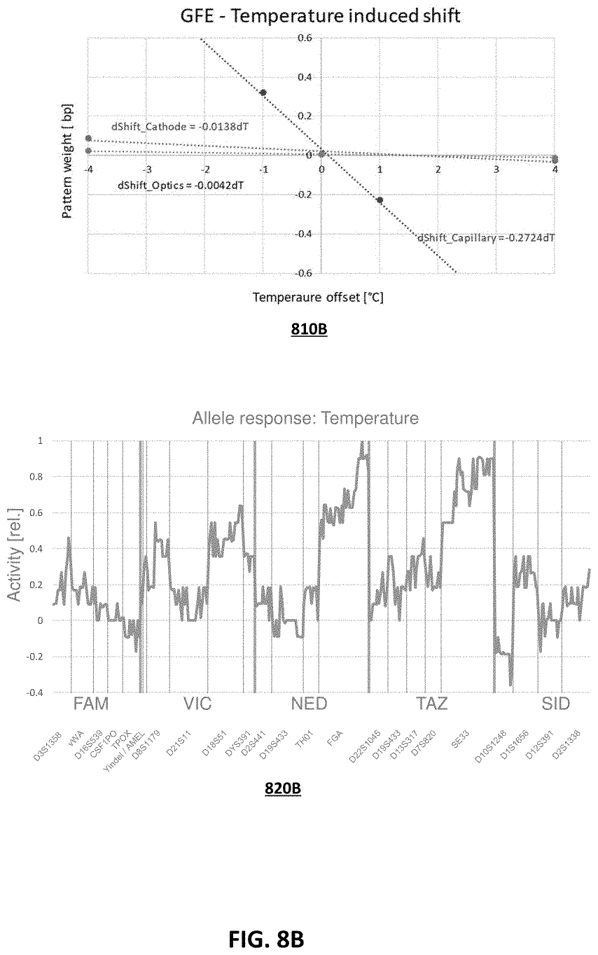

FIG. 8A illustrates exemplary experimental results for a gel degradation variable for an empirical migration model in accordance with an embodiment of the present invention. In graph 810A, the global response of the GFE (Global Filer Express) allelic ladder to gel degradation is shown. Separation current, plotted along the x-axis is used a proxy for gel degradation, and a higher current means that the gel is more degraded. In one embodiment of the invention, the gel is left in the instrument for a period of time, and allelic ladders are run at regular intervals using the same gel. For example, in one embodiment, an allelic ladder sample run is conducted once a day for several weeks, at room temperature (e.g., instrument coolers turned off), in order to increase the gel degradation speed.

The temperature in this experiment is held fixed. Experimentally, it can be shown in an embodiment of the present invention that the relationship between gel degradation and fragment size of each allele (also referred to as the pattern weight in number of base pairs, or bp) is linear within a certain range. The more degraded a gel is, the larger the shift in fragment sizing, and the molecule will appear larger in size. For example, looking at the global response behavior shown in graph 810A, it can be seen that the apparent fragment size of the allele having the strongest relative activity has shifted approximately one base pair when the gel has degraded such that separation current is 26 microamps, assuming a run at 18.2 microamps as a reference run where the pattern weight is 0 bp.

In graph 820A, the relative response of each allele in the allelic ladder to gel degradation is shown. Considering each of the peaks in the ladder, all other alleles will shift some percentage less than the allele having the peak measuring 1 on the y-axis of normalized relative activity values.

FIG. 8B illustrates experimental results for a temperature variable for an empirical migration model in accordance with an embodiment of the present invention. In graph 810B, the global response of the GFE (Global Filer Express) allelic ladder to temperature is shown to have a linear relationship, as shown when temperature is shifted three different instrument heaters represented in graph 810B, where the temperature shift in the capillary has the highest response. The gel degradation (e.g., separation current) in this experiment is held fixed. Experimentally, it can be shown in an embodiment of the present invention that the relationship between temperature and fragment size of each allele (also referred to as the pattern weight in number of base pairs, or bp) is linear within a certain range. Generally, (for GFE in combination with a specific selected ILS), the colder the temperature, the larger that the molecule will appear in size. Similarly, in graph 820B, the relative response of each allele in the allelic ladder to temperature is shown. As above, considering each of the peaks in the ladder, all other alleles will shift some percentage less than the allele having the peak measuring 1 on the y-axis of relative activity.

### Principal Component Analysis

When evaluating a fragment analysis electropherogram, the apparent sizes of a fragment, represented by a peak, is determined by interpolating the relative location of the peak to a set of reference peaks of known sizes, the internal lane standard (ILS). The determined size then, in turn, infers the number of base-pairs in the respective fragment, and jointly all fragments define a unique identity of the sample; in the field of HID implicating its source as one or several individuals. Unfortunately, the relative migration rate between the ILS and the fragment peaks varies, so the interpolated sizes will vary between runs even for a single sample run at different times. Hence the ‘lookup’ table, or ladder, for inferring the base-pair count cannot always be the same. Prior art approaches have provided a limited set of ladders, a ladder library, available on the system for the matching, i.e., selecting the ladder that matches any given sample the best.

For an otherwise fixed system, two parameters may determine the relative migration rates: how degraded—or ‘old’—the gel is and the gel temperature; a combination of the temperature of the capillary heater as assembled and controlled, and the environmental temperature, e.g., in a sunny window. It should be noted that other underlying physical factors may be driving these differences in migration, such as gel pore size and degree of denaturing of the amplified fragments, each of which is influenced by at least the above-mentioned parameters.

The influence of degradation and temperature are not the same. For instance, in one example (utilizing a GFE chemistry and an ILS used on Applied Biosystems RapidHIT™ ID instruments), a more degraded gel will make the peaks stemming from the loci D19S433 migrate relatively slower, making them appear larger. Temperature, on the other hand, virtually does not affect the migration of those specific fragments at all, relative to the ILS.

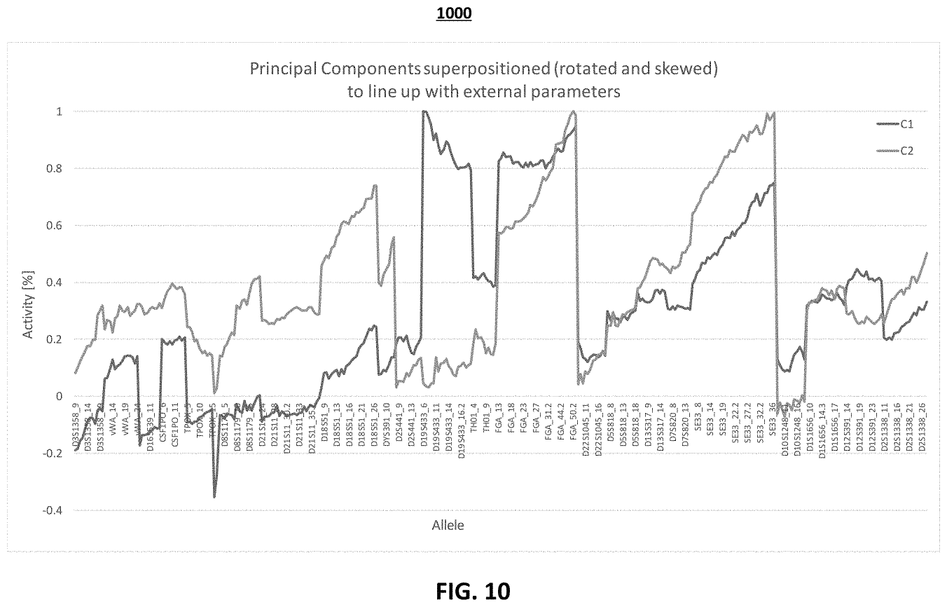

In general, the more degraded gel, or lower the temperature, the larger the apparent sizes—relative to the sizes of an imaginary run at a reference condition or under other ideal conditions. However, each fragment has a different response to each parameter. For the above example, as shown in graph 810B, or, e.g., component C2 of graph 1000 in FIG. 10 discussed below, if the temperature varies, long fragments of the loci D18S51 only shift ˜70% of what the long fragment peaks of FGA do, and there is a ˜50% difference in response between the short fragments and the long fragments of SE33. Some fragment peaks even shift in the other direction and appear shorter. The list of all these relative responses describes the ‘pattern’, or characteristic component, by which the migration is affected by the parameter.

So, for any given run, assuming that the exact conditions are known, the shifts for each of the peaks can be calculated by combining the two effects. Conversely, from the peak sizes from a sample run, a best-estimate can be made (since generally there will always be noise) of how much warmer or colder, or degraded the gel, that run was relative to the imaginary reference ideal run, and via that representative allelic ladder, also relative to any other run. To make the comparison via this representative allelic ladder, it is not necessary to have the same set of peaks, i.e., different samples can be used, with different sets of fragments, in the runs we compare. The imaginary reference run is discussed herein as the “representative allelic ladder, and can be thought of as comprising the ideal peak size for every imaginable fragment.

Over time, many sample runs are performed, all influenced by these two parameters. Even if it is not known a priori how much each of the parameters affected each run, one can use the data to find sets of responses (or ‘patterns’) that can best describe all the shifts in the population. One machine learning technique to do this is called Principal Component Analysis (PCA).

It is expected that a stable CE system should yield two significant PCA components, representing the aforementioned variations. A migration model of an embodiment of the present invention is based on the following decomposition: Decompose each ladder L̅_̅{̅I̅}̅ (the list bp's for each allele) into

$$\overline{{L}_{1}} = \overline{G} + {\sum }_{j = 1}^{n} {w}_{ij} \overline{{P}_{J}} + \overline{{δ}_{1}}$$

where G̅ is a ‘representative ladder’, P̅_{J} are the n different patterns (components; perturbations), and wᵢⱼ is how much of each pattern (j) contributes to each ladder (i), i.e., the weight—note that the weight for G (or P₀) is constrained to always be one. Finally, δ̅ₗ is any residue that cannot be described by the model (noise or undescribed patterns). In some embodiments of the present invention, n is a small number such as 2 or 3. Note that it is possible to define a model where G̅=0, but this typically this requires n to be incremented. There are multiple approaches to determining G̅ and the P̅_{J}s. One example is to use an experimental approach. Another example is to use historical reference data to determine G̅ and use such historical reference data in conjunction with PCA to determine the P̅_{J}s. Another example is to use other machine learning algorithms known to people skilled in the art.

It should be noted that other dimensionality reduction (or correlation finding) algorithms may be able to treat samples as incomplete ladders so that an effective model can be generated from test sample data without having to limit training data to data from runs of complete ladder samples. One approach for doing so is to force the residues of missing peaks to always be zero, and then find G̅ and P̅ⱼs that minimizes the total error. One benefit of this approach is that it allows training the model on larger data sets over time as instruments are used in the regular course of running new test samples.

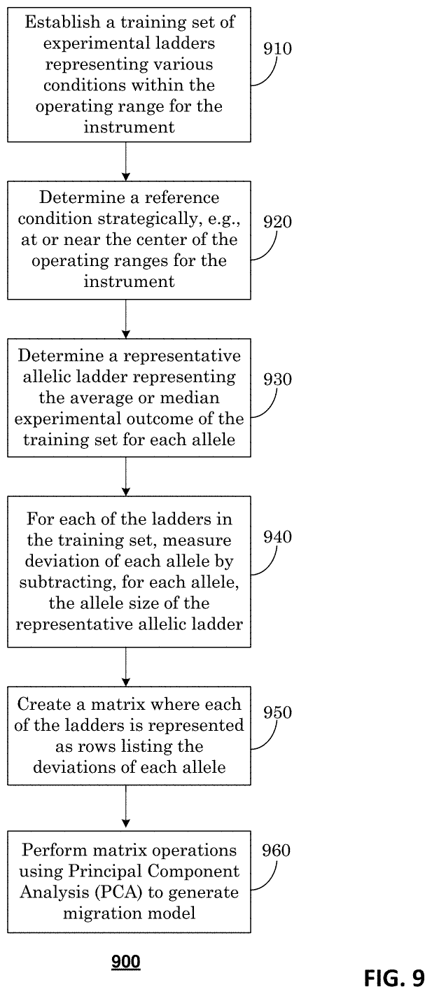

FIG. 9 illustrates a process for building a migration model based on PCA in accordance with an embodiment of the present invention. PCA is a technique used to emphasize variation and bring out strong patterns in a dataset. In one embodiment of the invention, PCA utilizes the properties of a correlation matrix to find principal components. Principal components are different from the characteristic components such as gel degradation and temperature mentioned above, in that the principal components describe the strongest dependencies in a data set rather than the change with any selected physical parameter. For example, for a dataset of five number series, the PCA algorithm will return five eigenvectors, with accompanying eigenvalues, which can be linearly recombined to reconstitute the full data set. However, and more importantly, if the number series correlate to one another, only a subset of the eigenvectors, those associated with the highest eigenvalues, need to be used if one can accept to reconstitute the dataset with small errors. As discussed above in an embodiment of the present invention, variations in apparent fragment size are found to be most significantly impacted by changes in temperature and gel degradation. Thus, in one embodiment of the invention, a PCA-based model having two principal components may be used.

The process to build a PCA-based migration model begins at step 910, where a training set of experimental ladders representing various conditions (e.g., temperature and gel degradation) within the operating range for the instrument. In the PCA-based migration model, the conditions for each ladder run do not need to be known. In addition, not all conditions need to be in the training set (or even close to all conditions), as the PCA-based migration model allows modeling those conditions when they are not in the training data. In one embodiment of the invention, a set of experimental ladders representing all (or as many as practicable) practical use cases, and hence representing all (or as many as practicable) of the various conditions, is used as the training set.

In step 920, a reference condition is determined strategically, e.g., at or near the center of the operating ranges for the instrument. Next, in step 930, a representative allelic ladder is determined to represent the average (or median) experimental outcome should many ladders be run at this reference condition. In one embodiment of the invention, the representative allelic ladder is determined to be the average or median experimental outcome of the training set for each allele. In some embodiments, one or more allelic ladders in the training set having the highest and lowest fragment size values for each allele might be discarded before calculating the average or median.

Other embodiments of the present invention utilize different methods for determining a representative allelic ladder. In one embodiment, an experiment is performed where many ladders are run at the reference condition, and the average sizes of each allele determined in this experiment is taken to be the representative allelic ladder. In another embodiment, a subset of the training set that centers around the reference condition is selected, and an average or median of the subset is taken to be the representative allelic ladder. In another embodiment, the single experimental ladder in the training set that most resembles the average ladder is determined to be the representative allelic ladder, or to select several experimental ladder that resemble the average ladder, and take the average of those to be the representative allelic ladder.

In step 940, for each of the ladders in the training set, the deviation of each allele is measured by subtracting, for each allele, the allele size of the representative allelic ladder. Then, in step 950, a matrix is created where each of the training set ladders is represented as rows listing the deviations for each allele. In step 960, the matrix operations of the principal component analysis (PCA) tool are performed to generate the PCA-based migration model. In one embodiment of the invention, MATLAB and other similar numerical computing tools and programming languages known to those skilled in the art can be used to perform the matrix operations of PCA and other statistical analysis described herein.

In another embodiment of the present invention, the representative allelic ladder may be deduced using PCA. A preliminary PCA-based migration model may be developed without calculating the deviation of each allele as set forth in step 940. In this embodiment, PCA is applied to determine preliminary components describing the data without the subtraction of any representative ladder. It is then determined how much of the strongest preliminary component needs to be used to reconstitute each of the ladders to the best square-fit approximation. Next, the median of these values is found, and each of the values in said strongest component are multiplied with that median value. This series of numbers is then used as the representative allelic ladder In another embodiment, it is possible to not specifically define a “representative ladder” at all, but rather use said preliminary PCA-based model as the final model. In this embodiment, the function of the “representative ladder” will be accommodated by the first component of the PCA analysis, and it is therefore recommended to expand the model to use three principal components rather than two.

FIG. 10 illustrates a graphical representation 1000 of two linear combinations of the two most significant principal components generated in a PCA-based migration model in accordance with an embodiment of the present invention. Note that any linear combination that can be constructed by the most significant two principal components returned from PCA output, can also be constructed from these two linearly combined components. Component C1 shows a perturbation that closely tracks the empirically identified perturbation associated with gel degradation, and C2 shows a perturbation that closely tracks the empirically identified perturbation associated with temperature changes. This similarity can be seen by comparing the graph of the two principal components in FIG. 10 with the experimental results shown in graph 820A in FIG. 8A (for gel degradation) and in graph 820B in FIG. 8B (for temperature changes). As previously discussed, the two strongest influencers for the variations in fragment sizing data are expected to be temperature changes and gel degradation.

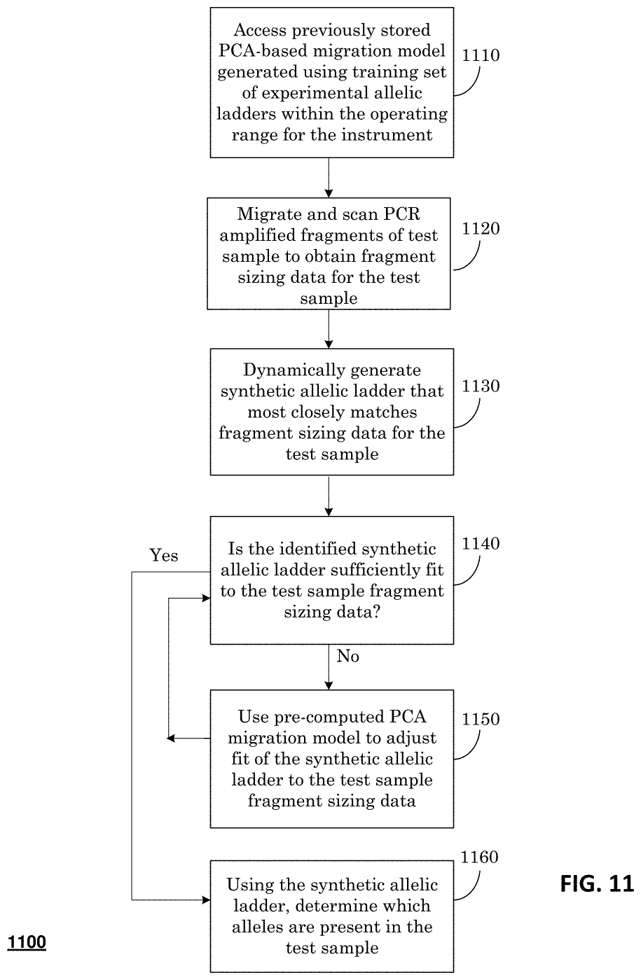

FIG. 11 illustrates a PCA-based STR analysis workflow process in accordance with an embodiment of the present invention where no reference sample run is required. In step 1110, a pre-computed PCA-based migration model generated using a training set of experimental allelic ladders within the operating range of the instrument is accessed. In step 1120, fragment sizing data for the test biological sample (e.g., buccal swab for suspect or victim human, crime scene sample) is obtained by migrating and scanning PCR amplified fragments of the test biological sample. In step 1130, a synthetic allelic ladder that matches fragment sizing data for the test sample is generated using the PCA-based migration model. In one embodiment, the synthetic allelic ladder is generated by selecting a ladder from a set of ladders, the set of ladders corresponding to sets of principal component values at regular intervals within a valid operating range. In another embodiment, the generated synthetic allelic ladder is randomly generated within a valid operating range of principal component values.

In step 1140, a determination is made as to whether the identified synthetic allelic ladder is sufficiently fit to the test sample fragment sizing data. In one embodiment of the invention, if the identified synthetic allelic ladder contains does not contain measurements that are within 0.10 bp for each allele in the test sample fragment sizing data, then the identified ladder is not sufficiently fit. In another embodiment, if the identified synthetic allelic ladder contains does not contain measurements that are within 0.35 bp for each allele in the test sample fragment sizing data, then the identified ladder is not sufficiently fit. If the answer to step 1140 is “Yes”, then in step 1160 the synthetic allelic ladder is used to determine which alleles are present in the test sample. If the answer in step 1140 is “No”, then in step 1150 the pre-computed PCA-based migration model is used to adjust the fit (by adjusting the weights in the model) of the synthetic allelic ladder to the test sample fragment sizing data. In one embodiment of the present invention, for a test sample where no synthetic ladder can be constructed having a sufficient fit, a mechanism to abort the process of finding a synthetic ladder that is a sufficient fit may be implemented (e.g., abort the process after a pre-determined number of iterations of adjustments has been reached).

In an embodiment of the present invention, there are two parts to achieve a sufficient fit. In the first part, a score for the fit is defined and an algorithm is used to optimize the fit. An example of an algorithm for adjusting and/or optimizing the weights of the model to generate a synthetic ladder to fit a test sample or ladder used in one embodiment of the invention is the Broyden-Fletcher-Goldfarb-Shanno Bounded (BFGS-B) algorithm available in the Math.NET toolkit. This algorithm is one of many possible optimization algorithms that can be used for this purpose. In this case, the algorithm will find a minimum of a function F(w₁, w₂) where w₁ and w₂ are the weights used in the model to reconstruct a synthetic ladder. The function F is defined such that a good fit returns a low number. The algorithm will test the function and find values for w₁ and w₂ that return optimized lowest numbers for the optimization function F. Optimization algorithms typically use additional parameters for the optimization. Examples of such parameters are the allowable range of w₁ and w₂. Another example is the accuracy by which it will determine the w₁ & w₂ values (e.g., parameter tolerance). One example of F is to, for each peak in a sample, find the nearest synthetic peak for the given w₁ & w₂; calculate the absolute difference in base pairs between said sample peak and said synthetic peak and return the arithmetic mean for all the peaks. Another example that allows for rare genotypes and the presence of unanticipated artifacts is to exclude the two largest differences before calculating said arithmetic mean. Another example is to use the sum of the absolute differences instead of said arithmetic mean.

In the second part it is determined how much optimizing is required before the fit is considered to be sufficient. In some embodiments of the present invention, for components that have been normalized such that their absolute maximum value is one, w₁ and w₂ can be optimized with a “parameter tolerance” of 0.35 bp or 0.1 bp or 0.01 bp. (=accuracy by which it will determine the w₁ & w₂ values—see above). This means that the algorithm will iterate until it ‘concludes’ it has determined the w₁ & w₂ that minimizes F to this tolerance; i.e., the theoretical minimum, should we optimize indefinitely, is within 0.35 bp or 0.1 bp or 0.01 bp of the returned values. For other absolute maximum values of the components, the parameter tolerance can be divided by this number to achieve the same effect. (If a weight is within 0.35 bp, this means—if the components are normalized to one—that the tolerance of the most active allele is 0.35 bp, all others are better.

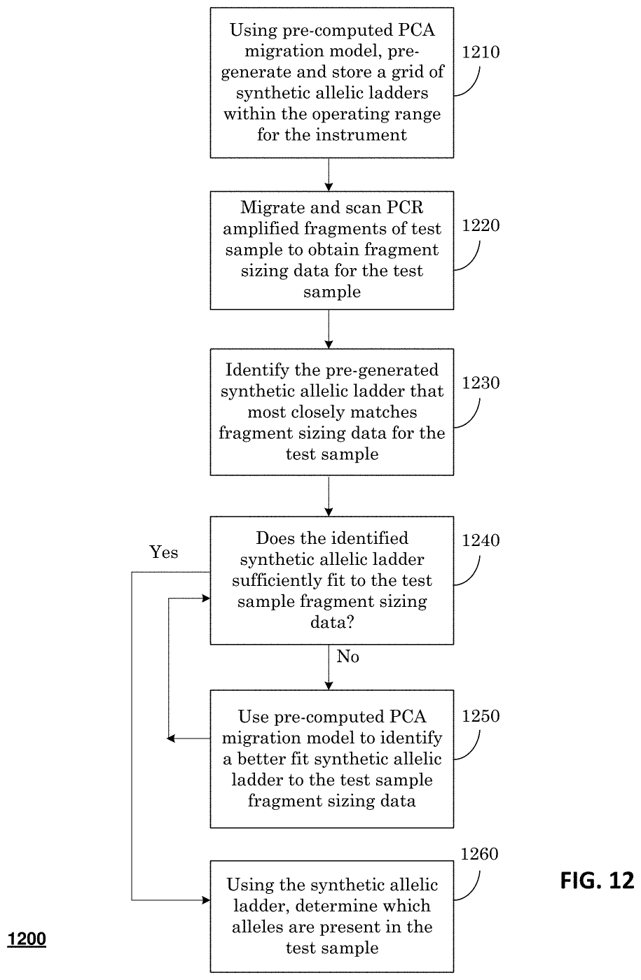

FIG. 12 illustrates a PCA-based STR analysis workflow process in accordance with another embodiment of the present invention, where again, no reference sample run is required. The process of FIG. 12 differs from the process of FIG. 11 in that a plurality of synthetic allelic ladders within the desired operating range for the instrument is pre-generated and stored. Having a pre-generated set of allelic ladders representative of the range of the principal components may reduce computational requirements in the STR analysis using the PCA-based migration model. Furthermore, although FIGS. 11 and 12 reference generating ladders from a PCA-created model, the steps of FIG. 11 and FIG. 12 apply to migration models generated via other disclosed methods.

In step 1220, fragment sizing data for the test biological sample (e.g., buccal swab for the subject, client, suspect or victim human; or crime scene sample) is obtained by migrating and scanning PCR amplified fragments of the test biological sample. In step 1230, a pre-generated and stored synthetic allelic ladder that most closely matches fragment sizing data for the test sample is identified. In one embodiment, a set of stored experimentally derived allelic ladders are included with the set of synthetic allelic ladders and a stored experimentally derived allelic ladder may be identified in place of a synthetic allelic ladder. In step 1240, a determination is made as to whether the identified synthetic allelic ladder is sufficiently fit to the test sample fragment sizing data. If the answer to step 1240 is “Yes”, then in step 1260 the identified synthetic (or stored native) allelic ladder is used to determine which alleles are present in the test sample. If the answer in step 1240 is “No”, then in step 1250 the pre-computed PCA-based migration model is used to adjust the fit of the synthetic allelic ladder to the test sample fragment sizing data until the fit is determined to be sufficient (or the process is aborted) as discussed above. In another embodiment, the density of the pre-stored ladders is such that the first identified synthetic (or native) allelic ladder is sufficiently fit to the test sample, and optimization steps 1240 and 1250 are not performed.

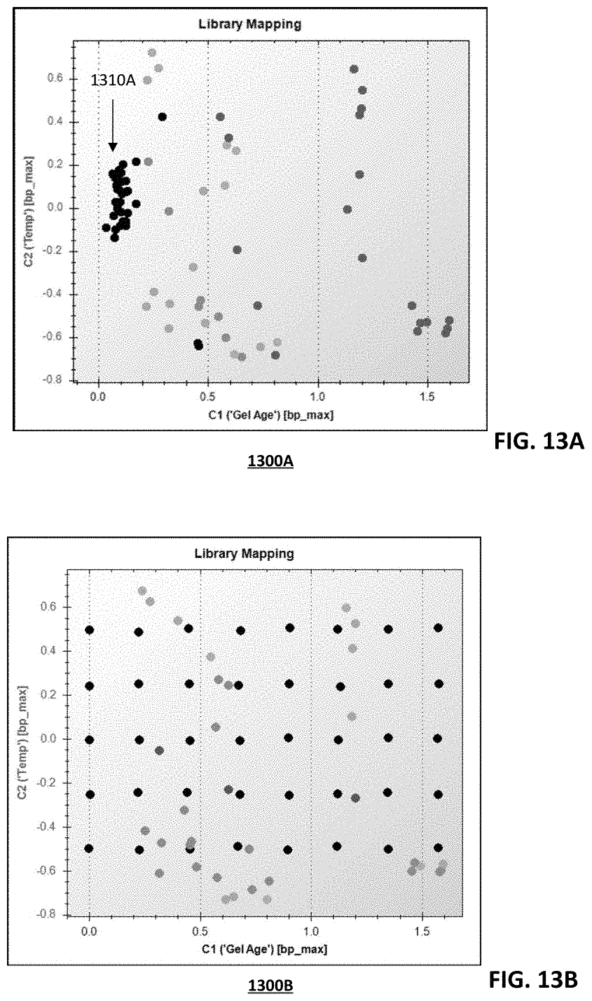

FIG. 13A illustrates a graphical representation of a PCA analysis of a ladder library. Graph 1300A shows a PCA analysis of a “naïve” (e.g., manually curated without particular attention to density or coverage area) ladder library showing the weights w₁ and w₂ for the respective components C1 and C2 corresponding to each ladder. In FIG. 13A, components C1 and C2 are linear combinations of the principal components derived from PCA analysis, where C1 is the component more associated with gel degradation. C2 is the component more associated with temperature changes. The black dots represent the allelic ladder library. The colored dots represent test sample runs. As shown in graph 1300A, the PCA analysis reveals that the allelic ladders in the naïve ladder library are largely clustered near a small range of component values shown at 1310A. Test samples that have weights, w₁ and w₂, of sufficiently fit synthetic ladders far from cluster 1310A are more likely to fail to generate a valid match to any of the ladders in the ladder library, as shown by red dots, whereas the green dots show a valid match. All ladders in the library can be well described with the two parameters.

In FIG. 13A, color may be used to indicate a largest deviation (model error+noise) for a particular test sample, for example: Red=Failed match; Yellow=0.35-0.5 bp; while all shades of green=less model error+noise, and valid match.

FIG. 13B illustrates a graphical representation of a PCA analysis of a synthetic ladder library in accordance with an embodiment of the present invention. Graph 1300B shows a PCA analysis of a synthetically generated ladder library showing the weights, w₁ and w₂, for the respective components C1 and C2 corresponding to each ladder. C1 is the component more associated with gel degradation. C2 is the component more associated with temperature changes. The black dots in graph 1300B represent the synthetic allelic ladder library. The colored dots represent test sample runs. As shown in graph 1300B, the PCA analysis shows that the synthetic ladder library comprises ladders at regular intervals along the range of principal component values, and thus shows that the synthetically generated ladder library offers more coverage over the full range of operating conditions than the “naïve” ladder library. Graph 1300B shows that the synthetic ladder library not only confirms the valid test sample runs of the “naïve” ladder library, but also has potentially improved accuracy of the instrument, as more sample runs outside the principal component ranges covered by the “naïve” ladder library generated valid matches.

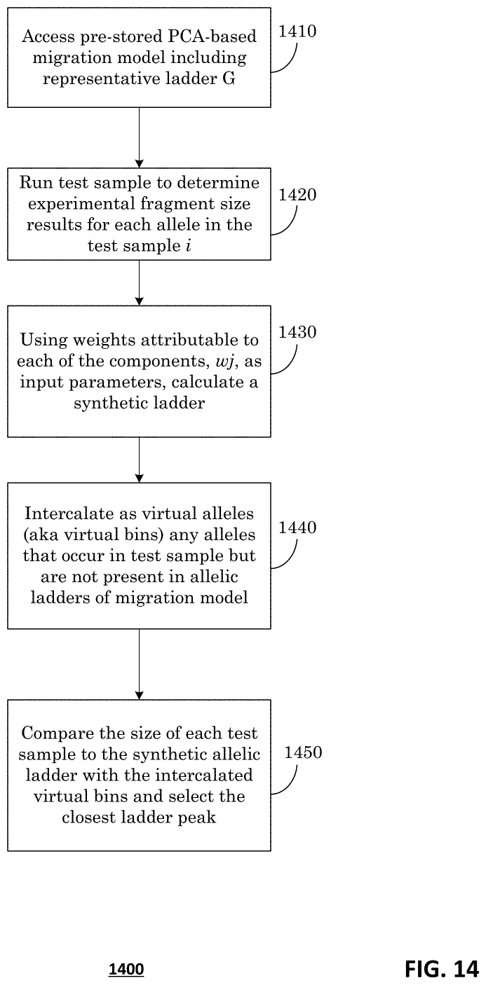

FIG. 14 illustrates a process for generating a synthetic allelic ladder, from the migration model (PCA or experimentally or otherwise constructed), and comparing said synthetic ladder with a test sample, in accordance with an embodiment of the present invention. In step 1410, a pre-stored migration model including representative ladder G, and perturbation vectors (or ‘components’) Pj, is accessed. In some embodiments of the present invention, the number of components, n, is small such as 2, or 3. In step 1420, a test sample is run in the analysis instrument to determine experimental fragment size results for each allele present in the test sample.

In step 1430, weights attributable to each of the components, wⱼ, are used as input parameters and a synthetic ladder is calculated using the following formula

$$\overline{{L}_{Syntetic}} = \overline{G} + {\sum }_{j = 1}^{n} {w}_{j} \overline{{P}_{J}}$$

In step 1440, any virtual alleles (also referred to as virtual bins) that may occur in the test sample, but not found in the migration model are intercalated. The expected position of these virtual alleles may be interpolated or extrapolated from the expected size of the alleles present in the allelic ladders of the migration model. In step 1450, the size of each sample peak is compared to the peaks in the synthetic ladder with the intercalated virtual bins. The ladder peak having the smallest difference in size to the sample peak is selected, however only peaks associated with the same dye color as the sample peak are considered. From the collection of smallest differences, a match error is calculated. The match error is a scalar that reflects how well the synthetic ladder and the sample matches. One example of how the match error may be calculated is to take the arithmetic mean of said all smallest differences. Another example is to exclude the two largest of said smallest differences before calculating said arithmetic mean. This can accommodate for rare genotypes not included among the virtual bins, as well as the presence of unanticipated artifact peaks in the test sample. Another example is to use the sum of the absolute differences instead of said arithmetic mean.

Reconstituting a ladder may be considered the idea of finding wᵢⱼ such that the total difference between the resulting number series and the allele sizes of an experimental ladder (or test sample) is as small as possible, where said total difference is the sum of the square of the difference for each of the alleles. When reconstituting a ladder and the total difference is small, the model can be said to describe the ladder well. If a large dataset can be reconstituted with only minor errors, as defined by statistical means such as median, standard deviation, and max error, the model can be said to be accurate.

It is conceivable to identify additional variables and to expand the model with their characteristic components, or to incorporate more of the principal components returned from the PCA algorithm into the model. The model will be more accurate, with each component properly implemented. However, in some embodiments of the present invention discussed here, two principal components are enough to provide modeling of a stable system at relevant accuracy, although other embodiments may use three or more principal components.

FIG. 15 illustrates an exemplary PCA-based migration model 1500 in accordance with an embodiment of the present invention, used here to reconstruct a given allelic ladder. From a set of allelic ladder sample runs 1510, a representative ladder 1520 is determined for each of the alleles in sample runs 1510. Here representative ladder 1520 is shown for each first seven alleles, which are labeled as Alleles 1-7. Next, PCA analysis is performed on the set of allelic ladder sample runs 1510 to generate principal components (patterns) P₁ and P₂ for each allele, as shown at 1531 and 1532. The set of weights wᵢⱼ, e.g., how much of each pattern (j) contributes to the ladder subject to reconstruction (i) is calculated using the methods described above, and shown in bold text on white background at column 1540. Using these values, the reconstructed allelic ladder can be calculated as shown at 1550. Other ladders can be generated from the same model by varying the weight values in column 1540. As noted earlier, components C₁ and C₂, constructed as linear combinations of P₁ and P₂, can be equivalently used.

In one embodiment, the migration model (such as a PCA-based migration model) stored or accessed by the instrument may be systematically improved upon over time based on machine learning of sample run data. In an embodiment, other “correlation-finding” (otherwise known as “dimensionality reduction”) algorithms known in the art may be used to build migration models in a manner similar to the PCA-based migration model discussed above. In addition to PCA, such approaches may include Non-negative Matrix Factorization (NMF), Kernel PCA, Graph-based Kernel PCA, Linear Discriminant Analysis (LDA), Generalized Discriminant Analysis (GDA), and Autoencoder, among others. Such “correlation finding” algorithms may be able to utilize incomplete ladders (such as those ladders resulting from test sample runs) to develop the migration model. In one embodiment, the migration model may be adjusted using external adjustments, e.g., by adding an offset to the representative ladder so the model fits test samples better than complete ladders. This may be because the test samples may have a systematic offset, meaning that the test samples migrate differently than how allelic ladder samples migrate. An offset can be made to compensate for this difference in migration behavior, so that the sample alleles may migrate on average with a zero deviation, whereas allelic ladders may have a non-zero deviation. Such an offset may be determined by, e.g., analyzing a large data set of test sample runs with the migration model, and finding statistical deviations. In another embodiment, the migration model may be adjusted using internal adjustments, e.g., by making linear combinations of migration model components and reference (or representative ladders) that are better aligned with physical realities (e.g., combinations of gel degradation (e.g., gel age) and temperature that realistic operating conditions).

A PCA-based migration model and synthetic allelic ladder library as discussed in accordance with embodiments of the present invention can have several uses, including:
Confirming that any specific run can be described at high quality by the model such that it increases the confidence the run was not compromised.
Monitor the operating conditions of an instrument to confirm it is operating within the approved range.
Confirming that other system parameters affecting migration other than temperature and gel degradation are held constant. In particular, as parts of the system is being altered such as gel and capillary replacements, as well as for quality control during manufacturing of gel, cartridges, capillary replacements, and other consumables.
Synthetically generating noise free reference runs (for the ladder library)
Performing allelic ladder free analysis

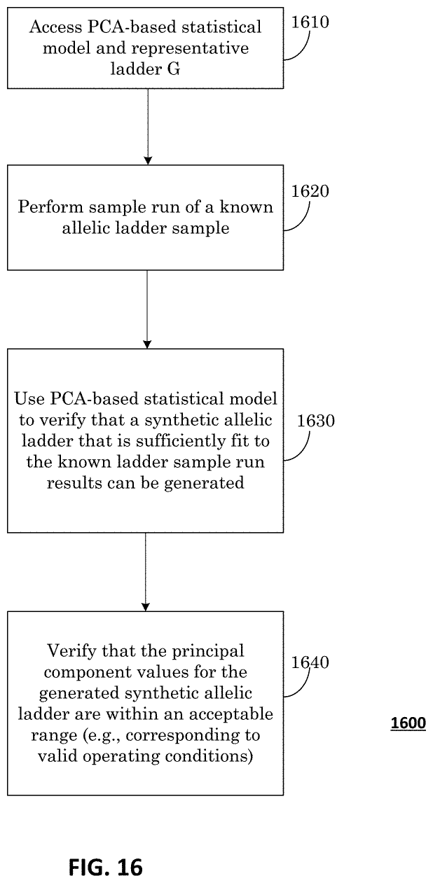

FIG. 16 illustrates a PCA-based CE instrument validation process using synthetic allelic ladders in accordance with an embodiment of the present invention. In step 1610, the PCA-based statistical model and representative ladder G are accessed. In step 1620, a sample run of a known allelic ladder sample is performed on the CE instrument to be validated. In step 1630, the PCA-based statistical model is used to verify that a synthetic allelic ladder that is sufficiently fit to the known allelic ladder sample run results can be generated. In step 1640, the principal component weights for the generated synthetic allelic ladder are used to verify that the principal component weights for the generated synthetic allelic ladder are within an acceptable range (e.g., corresponding to valid operating conditions). This can be verified by limiting how much each of the patterns can be used to fit the sample data. In some embodiments of the present invention, a similar process can also be used to verify instrument performance for quality control during manufacturing of gels, capillaries and cartridges. In some embodiments of the invention, the known allelic ladder sample run results that deviate from the model less than 0.1 bp, 0.15 bp, or 0.35 bp, for example, may indicate that the instrument operation is valid. Other aggregates of the differences between the ladders can be used as validating metrics. In one embodiment of the present invention, a sample is used instead of the known allelic ladder sample, and its weights are determined by finding a synthetic allelic ladder with an optimized or sufficient fit. The operation of the instrument can be deemed valid should no peak deviate more than, e.g., 0.1 bp, 0.15 bp, or 0.35 bp from said synthetic ladder.

The migration models in embodiments of the present invention described above can be used to analyze how well an actual ladder fits a ladder generated by the model. For example, it may be desirable for an allelic ladder library to contain ladders that are representative of the normal behavior at all various circumstances a run may be performed at. By analyzing historical data using the model in accordance with the present invention, it is possible to make informed decisions of which ladders to include in an allelic ladder library. A model, preferably one that captures well the behavior of the instrument, can identify sample and ladder runs that are less conformant to the model. An example of non-conformance could be a peak that has been distorted by optical noise such that its peak has been shifted and therefore assigned an inaccurate size. It is preferred to not represent such non-systematic events in the ladder library. In some embodiments of the invention, well-conforming ladders have no peaks that deviate from the model more than 0.1 bp, 0.15 bp, or 0.35 bp, for example. This deviation can be referred to as maximum (max) deviation. A synthetic allelic ladder that has been generated by the model is expected to have a max deviation of zero, or at least no larger a deviation than by which numbers are rounded during analysis, 0.05 bp or 0.1 bp.

If a large amount of sample and ladder data is analyzed using the model, it can be determined how each allele distributes from the theoretical model (i.e. for each sample, find the best ladder using the theoretical model, determine how much each allele differs from it (deviation of sample peak from model peak), then collect the statistics from all samples for each allele.) In one embodiment of the invention, each distribution of deviations of peaks from the model should center close to zero, e.g., better than 0.1 bp; and the corresponding 3 sigma (3 standard deviations) should be low, e.g., 0.15 bp. Approximating the distributions with a Gaussian distribution, this means that more than 99% of peaks called at an allele with the aforementioned distribution will be within 0.25 bp.

In one embodiment of the invention as discussed above, a static (pre-selected and/or pre-calculated) ladder library with a specified density level is constructed and stored on the analysis instrument or system. This static library may be searched prior to generating a synthetic ladder, and may be more efficient in situations where computational resources are constrained such as dynamically generating one or more synthetic ladders “on the fly” is not efficient or feasible. In one embodiment of the present invention, a ladder library comprises a plurality of ladders having w₁ and w₂ values that are spaced within approximately 0.2 bp apart across the range of valid operating values for the system. For a static (pre-selected and/or pre-calculated) ladder library with a discrete set of ladders, when determining the best ladder to fit a test sample, the theoretically ideal optimal ladder that the model could reconstitute may not be present. But if the ladders in the library have been selected such that there is at least one ladder for each 0.2 bp interval of w₁ and w₂, respectively, there will always be at least one ladder available that is no more than about 0.1 bp ‘away’ from each of the weights of said ideal ladder. If the ladders in the library have non-conformity no larger than 0.1 bp, a sample deviating 0.25 bp can in total not deviate more than about 0.45 bp for the most active allele (max deviation). This max deviation is determined as follows: as it can be experimentally found that the most active allele (possible worst case) may deviate 0.25 bp from the theoretical ideal ladder due to noise and systemic variations, adding 0.1 bp deviation due to 0.2 bp interval density of the static ladder library discussed above, and 0.1 bp deviation due to noise in the library ladder, a total maximum deviation of 0.45 bp results. While these numbers are intended as an illustrative example, higher density or lower density libraries may be constructed. Higher density libraries will reduce the likelihood of failed matches, but computational and storage limitations (e.g., for analysis software) may be a constraint. Conversely, a lower density library may be used in lower computational power systems but the likelihood of failed or incorrect matches is higher. The exact calculations will depend on the relation between the components should the deviation be off on more than one of the w₁ or w₂ values. In one embodiment of the invention as noted above, experimental data has indicated that when the deviation is larger than, for example 0.45 bp or 0.5 bp, a peak may be incorrectly called.

Historical ladders can be assigned w₁ and w₂ values by minimizing the match error. A synthetic ladder can be created using these w₁ and w₂ values and the maximum deviation for any allele between said historical ladder and said synthetic ladder is a metric of how non-conforming said historical ladder is. By identifying the w₁ and w₂ of well-conforming historical ladders (e.g. having a maximum deviation of no more than 0.1 bp, 0.15 bp, or 0.35 bb), and/or creating synthetic ladders from selected w₁ and w₂ values, it is possible to, in an informed manner, gather a ladder library, designed to have a sufficient density, d, across a range of w₁ and w₂, where the density, d, is defined such there is no combination of and w₂′ within said range where there is no ladder in the ladder library for which |w₁−w₁′|<d and |w₂−w₂′|<d (and so forth should there be more dimensions). Note that it is possible to define different densities for different dimensions. For the specific circumstances and statistics discussed in the previous illustrative example, it is suggested that a ladder density of 0.2 bp or lower would be sufficient to, with high probability, cover all run conditions on a (non-defective) instrument across the full range of operation. Please refer to FIG. 13B for an illustration of such a designed library.

For validation of a designed ladder library, a large amount of sample and ladder data can be analyzed using the designed ladder library, and it can be determined how said data, for each of the alleles, distributes from the ladder library. In one embodiment of the present invention, for a ladder library the distribution of deviations for each allele should center close to zero, e.g., within 0.1 bp; and the corresponding 3 sigma (3 standard deviations) should be low, e.g. 0.35 bp or lower.

### EXEMPLARY COMPUTING DEVICE EMBODIMENT

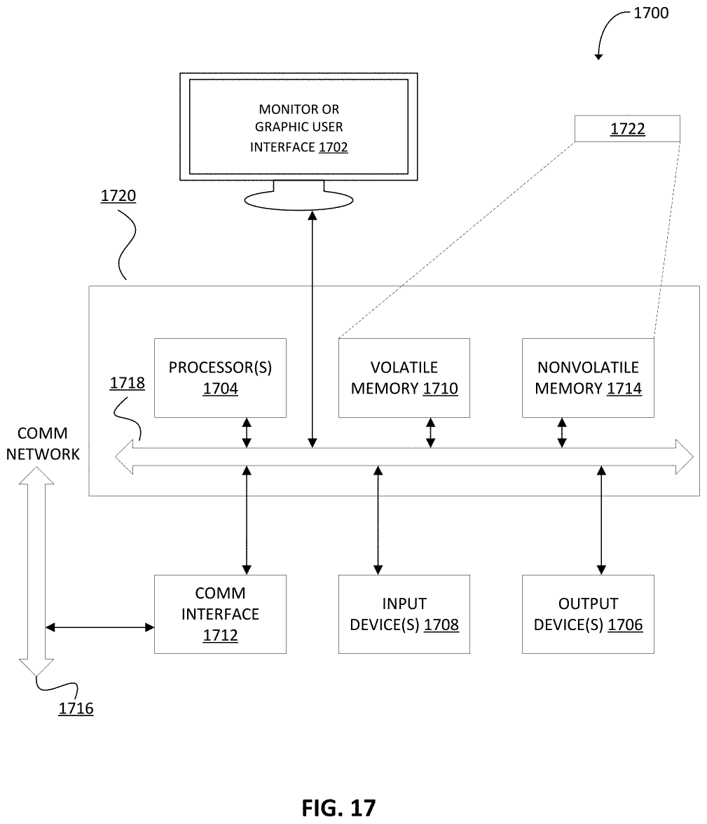

FIG. 17 is an example block diagram of a computing device 1700 that may incorporate embodiments of the present invention. FIG. 17 is merely illustrative of a machine system to carry out aspects of the technical processes described herein, and does not limit the scope of the claims. One of ordinary skill in the art would recognize other variations, modifications, and alternatives. In one embodiment, the computing device 1700 typically includes a monitor or graphical user interface 1702, a data processing system 1720, a communication network interface 1712, input device(s) 1708, output device(s) 1706, and the like.

As depicted in FIG. 17, the data processing system 1720 may include one or more processor(s) 1704 that communicate with a number of peripheral devices via a bus subsystem 1718. These peripheral devices may include input device(s) 1708, output device(s) 1706, communication network interface 1712, and a storage subsystem, such as a volatile memory 1710 and a nonvolatile memory 1714. The volatile memory 1710 and/or the nonvolatile memory 1714 may store computer-executable instructions and thus forming logic 1722 that when applied to and executed by the processor(s) 1704 implement embodiments of the processes disclosed herein.

The input device(s) 1708 include devices and mechanisms for inputting information to the data processing system 1720. These may include a keyboard, a keypad, a touch screen incorporated into the monitor or graphical user interface 1702, audio input devices such as voice recognition systems, microphones, and other types of input devices. In various embodiments, the input device(s) 1708 may be embodied as a computer mouse, a trackball, a track pad, a joystick, wireless remote, drawing tablet, voice command system, eye tracking system, and the like. The input device(s) 1708 typically allow a user to select objects, icons, control areas, text and the like that appear on the monitor or graphical user interface 1702 via a command such as a click of a button or the like.

The output device(s) 1706 include devices and mechanisms for outputting information from the data processing system 1720. These may include the monitor or graphical user interface 1702, speakers, printers, infrared LEDs, and so on as well understood in the art.

The communication network interface 1712 provides an interface to communication networks (e.g., communication network 1716) and devices external to the data processing system 1720. The communication network interface 1712 may serve as an interface for receiving data from and transmitting data to other systems. Embodiments of the communication network interface 1712 may include an Ethernet interface, a modem (telephone, satellite, cable, ISDN), (asynchronous) digital subscriber line (DSL), FireWire, USB, a wireless communication interface such as Bluetooth or WiFi, a near field communication wireless interface, a cellular interface, and the like. The communication network interface 1712 may be coupled to the communication network 1716 via an antenna, a cable, or the like. In some embodiments, the communication network interface 1712 may be physically integrated on a circuit board of the data processing system 1720, or in some cases may be implemented in software or firmware, such as “soft modems”, or the like. The computing device 1700 may include logic that enables communications over a network using protocols such as HTTP, TCP/IP, RTP/RTSP, IPX, UDP and the like.

The volatile memory 1710 and the nonvolatile memory 1714 are examples of tangible media configured to store computer readable data and instructions forming logic to implement aspects of the processes described herein. Other types of tangible media include removable memory (e.g., pluggable USB memory devices, mobile device SIM cards), optical storage media such as CD-ROMS, DVDs, semiconductor memories such as flash memories, non-transitory read-only-memories (ROMS), battery-backed volatile memories, networked storage devices, and the like. The volatile memory 1710 and the nonvolatile memory 1714 may be configured to store the basic programming and data constructs that provide the functionality of the disclosed processes and other embodiments thereof that fall within the scope of the present invention. Logic 1722 that implements embodiments of the present invention may be formed by the volatile memory 1710 and/or the nonvolatile memory 1714 storing computer readable instructions. Said instructions may be read from the volatile memory 1710 and/or nonvolatile memory 1714 and executed by the processor(s) 1704. The volatile memory 1710 and the nonvolatile memory 1714 may also provide a repository for storing data used by the logic 1722. The volatile memory 1710 and the nonvolatile memory 1714 may include a number of memories including a main random access memory (RAM) for storage of instructions and data during program execution and a read only memory (ROM) in which read-only non-transitory instructions are stored. The volatile memory 1710 and the nonvolatile memory 1714 may include a file storage subsystem providing persistent (non-volatile) storage for program and data files. The volatile memory 1710 and the nonvolatile memory 1714 may include removable storage systems, such as removable flash memory.

The bus subsystem 1718 provides a mechanism for enabling the various components and subsystems of data processing system 1720 communicate with each other as intended. Although the communication network interface 1712 is depicted schematically as a single bus, some embodiments of the bus subsystem 1718 may utilize multiple distinct busses.

It will be readily apparent to one of ordinary skill in the art that the computing device 1700 may be a device such as a smartphone, a desktop computer, a laptop computer, a rack-mounted computer system, a computer server, or a tablet computer device. As commonly known in the art, the computing device 1700 may be implemented as a collection of multiple networked computing devices. Further, the computing device 1700 will typically include operating system logic (not illustrated) the types and nature of which are well known in the art.

One embodiment of the present invention includes systems, methods, and a non-transitory computer readable storage medium or media tangibly storing computer program logic capable of being executed by a computer processor.

Those skilled in the art will appreciate that computer system 1700 illustrates just one example of a system in which a computer program product in accordance with an embodiment of the present invention may be implemented. To cite but one example of an alternative embodiment, execution of instructions contained in a computer program product in accordance with an embodiment of the present invention may be distributed over multiple computers, such as, for example, over the computers of a distributed computing network.

While the present invention has been particularly described with respect to the illustrated embodiments, it will be appreciated that various alterations, modifications and adaptations may be made based on the present disclosure and are intended to be within the scope of the present invention. While the invention has been described in connection with what are presently considered to be the most practical and preferred embodiments, it is to be understood that the present invention is not limited to the disclosed embodiments but, on the contrary, is intended to cover various modifications and equivalent arrangements included within the scope of the underlying principles of the invention as described by the various embodiments referenced above and below.

### Terminology

Terminology used herein with reference to embodiments of the present invention disclosed in this document should be accorded its ordinary meaning according to those of ordinary skill in the art unless otherwise indicated expressly or by context.

“Allelic ladder” or “allelic ladder data” refers herein to the fragment sizing data set for an allelic ladder sample run on a CE instrument.

“Allelic ladder sample” refers to a calibration sample that includes a collection of known STR alleles that the CE instrument is testing for, and generally comprises a large number (e.g., several hundred) known STR alleles.

“Synthetic allelic ladder” or “synthetic allelic ladder data” refers to allelic ladder data that has been generated from a model rather than from an actual run of an allelic ladder sample.

“Capillary electrophoresis genetic analyzer” or “capillary electrophoresis DNA analyzer” in this context refers to an instrument that applies an electrical field to a capillary loaded with a biological sample so that the negatively charged DNA fragments move toward the positive electrode. The speed at which a DNA fragment moves through the medium is roughly inversely proportional to its molecular weight. This process of electrophoresis can separate the extension products by size, preferably at a resolution of one base or less.

“Exemplary commercial CE devices” in this context may refer to and include, but are not limited to, the following: the Applied Biosystems, Inc. RapidHIT™ ID System (single capillary) and RapidHIT™ 200 System (8 capillary); the Applied Biosystems, Inc. (ABI) genetic analyzer models 310 (single capillary), 3130 (4 capillary), 3130xL (16 capillary), 3500 (8 capillary), 3500xL (24 capillary); the ABI SeqStudio genetic analyzer models; the ABI DNA analyzer models 3730 (48 capillary), and 3730xL (96 capillary); as well as the Agilent 7100 device, Prince Technologies, Inc.'s PrinCE™ Capillary Electrophoresis System, Lumex, Inc.'s Capel-105™ CE system, and Beckman Coulter's P/ACE™ MDQ systems, among others.

“Base pair” in this context refers to complementary nucleotides in a DNA sequence. Thymine (T) is complementary to adenine (A) and guanine (G) is complementary to cytosine (C).

What is claimed is:

1. A method of testing a biological sample comprising deoxyribonucleic acid (DNA) molecules for presence of a plurality of alleles, wherein DNA fragments obtained using the biological sample and corresponding to different alleles of the plurality of alleles have different fragment sizes, the method comprising:
obtaining test fragment sizing data by migrating and scanning, using an analysis instrument, a plurality of labelled DNA fragments corresponding to the biological sample;
using a pre-computed model to dynamically generate one or more first synthetic allelic ladders, the pre-computed model based on analysis of a plurality of fragment sizing data sets obtained from a plurality of previously conducted sample runs using either the same analysis instrument or using another comparable analysis instrument to measure fragment sizes;
determining whether the one or more first synthetic allelic ladders fits the test fragment sizing data sufficiently for identifying which of the plurality of alleles are present in the biological sample;
if the determination is that the one or more first synthetic allelic ladders does not fit the test fragment sizing data sufficiently, then generating one or more additional synthetic allelic ladders based on varying one or more parameters of the pre-computed model and determining whether any of the one or more additional synthetic allelic ladders fits the test fragment sizing data sufficiently for identifying which of the plurality of alleles are present in the biological sample; and
once a sufficiently fitting synthetic allelic ladder is identified, using the sufficiently fitting synthetic allelic ladder to determine which of the plurality of alleles are present in the biological sample.

2. The method of claim 1, wherein the analysis instrument comprises a capillary electrophoresis (CE) instrument.

3. The method of claim 1, wherein the plurality of previously conducted sample runs comprises one or more allelic ladder sample runs.

4. The method of claim 1, wherein the plurality of previously conducted sample runs comprises one or more one or more test sample runs from other biological samples.

5. The method of claim 1, wherein the one or more additional synthetic allelic ladders are generated after a sufficiently fitting allelic ladder is identified, in order to satisfy one or more optimization criteria.

6. The method of claim 1, wherein the pre-computed model is based on principal component analysis (PCA).

7. The method of claim 6, wherein the principal component analysis comprises determining a first principal component having a first principal component range, and a second principal component having a second principal component range.

8. The method of claim 7, wherein the principal component analysis further comprises determining a representative allelic ladder comprising a plurality of alleles, each associated with a representative fragment size, wherein the representative allelic ladder is associated with a set of reference conditions.

9. The method of claim 8, wherein determining the representative allelic ladder further comprises:
running a plurality of experimental sample runs on allelic ladder samples under the set of reference conditions; and
calculating the average fragment size of each of the plurality of alleles in the experimental sample runs.

10. The method of claim 8, wherein determining the representative allelic ladder further comprises:
selecting a subset of the plurality of fragment sizing data sets that are within a specified range of the set of reference conditions; and
calculating the average fragment size of each of the plurality of alleles.

11. The method of claim 8, wherein the determining the representative allelic ladder further comprises: generating a preliminary migration model without determining a representative allelic ladder, wherein the preliminary migration model generates a representative synthetic allelic ladder corresponding to the set of reference conditions.

12. The method of claim 8, further comprising finding a fragment sizing data set of the plurality of fragment sizing data sets that is a sufficient fit to the representative synthetic allelic ladder.

13. The method of claim 8, further comprising:
finding a subset of the plurality of fragment sizing data sets, wherein each fragment sizing data set in the subset comprises a sufficient fit to the representative allelic ladder; and
calculating an average fragment size for each of the alleles in the subset.

14. The method of claim 8, further comprising linearly combining the first and second principal components to align with a temperature component and a gel degradation component, and setting a first reference condition at a center value of the temperature component, and setting a second reference condition at an upper value of the gel degradation component.

15. The method of claim 8, further comprising:
for each of the plurality of fragment sizing data sets, calculating a deviation value for each allele in the fragment sizing data set by subtracting the reference fragment size value from the data set fragment size value;
storing a matrix comprising the deviation values for the plurality of fragment sizing data sets; and
performing one or more principal component analysis matrix operations to determine principal components.

16. The method of claim 1, wherein the pre-computed model comprises an empirical model generated by:
defining a first variable and a second variable wherein the first variable and the second variable impact migration in the pre-computed model;
determining a first experimental range for the first variable and a second experimental range for the second variable;
selecting a reference condition within the first and second experimental ranges;
conducting a first series of calibration sample runs across the first experimental range for the first variable while holding the second variable constant at the reference condition, and a second series of calibration sample runs across the second experimental range for the second variable while holding the second variable constant at the reference condition;
defining a first parameter for the first variable and a second parameter for the second variable such that the first and second parameters are zero at the reference condition; and the first parameter comprises a non-zero value at a deviation of the first variable from the reference condition, and the second parameter comprises a non-zero value at a deviation of the second variable from the reference condition;
for the first and second variables, determining regression parameters and aggregating a slope of each allele in first and second plots to generate a first characteristic component and a second characteristic component; and
generating a reference ladder by aggregating the intercepts for the slopes of each of the alleles in the calibration sample.

17. The method of claim 1 further comprising:
prior to using the pre-computed model to dynamically generate one or more first synthetic allelic ladders, first determining whether a pre-stored allelic ladder fits the test fragment sizing data sufficiently for identifying which of the plurality of alleles are present in the biological sample, the pre-stored allelic ladder comprising a fragment sizing data set obtained from one or more sample runs previously conducted on allelic ladder samples using either the same CE instrument or using another comparable CE instrument to measure fragment sizes, and
if the pre-stored allelic ladder is sufficiently fit, using the sufficiently fitting pre-stored allelic ladder to determine which of the plurality of alleles are present in the biological sample without generating any first or additional synthetic allelic ladders.

18. A deoxyribonucleic acid (DNA) analysis instrument comprising:
a capillary electrophoresis (CE) genetic analyzer comprising:
a sample port operable to receive a test biological sample comprising one or more DNA molecules, wherein the DNA molecule comprises one or more DNA loci and each DNA locus is associated with a plurality of alleles;
a thermal cycler connected to the sample port comprising a polymerase chain reaction (PCR) chamber operable to perform DNA amplification of DNA fragments of the test biological sample;
at least one CE capillary connected to the thermal cycler operable to receive and separate the amplified DNA fragments of the test biological sample; and
an optical detector operable to scan the CE capillary to detect fluorescent values of the amplified DNA fragments of the test biological sample; and
a signal processor connected to the optical detector and operable to generate test fragment sizing data corresponding to fluorescent values of the amplified DNA fragments of the test biological sample; and
a DNA profile generator connected to the CE genetic analyzer comprising:
a pre-computed model to dynamically generate a first synthetic allelic ladder, the pre-computed model having been derived based on statistical analysis of a plurality of fragment sizing data sets obtained from a plurality of sample runs previously conducted on allelic ladder samples using either the same CE instrument or using another comparable CE instrument to measure fragment sizes;
a fitter to determine whether the first synthetic allelic ladder fits the test fragment sizing data sufficiently for identifying which of the plurality of alleles are present in the biological sample, and if the fit is not sufficient, then signaling the pre-computed model to generate one or more additional synthetic allelic ladders based on varying one or more parameters of the pre-computed model and determining whether any of the one or more additional synthetic allelic ladders fits the test fragment sizing data sufficiently for identifying which of the plurality of alleles are present in the biological sample; and
an allele caller to determine which of the plurality of alleles are present in the biological sample once a sufficiently fitting synthetic allelic ladder is identified.

19. The DNA analysis instrument of claim 18, wherein the DNA profile generator further comprises a database storing the plurality of fragment sizing data sets obtained from the plurality of sample runs previously conducted on allelic ladder samples using either the same CE instrument or using another comparable CE instrument to measure fragment sizes.

20. The DNA analysis instrument of claim 18, wherein the DNA profile generator remotely accesses the plurality of fragment sizing data sets obtained from a plurality of sample runs previously conducted on allelic ladder samples using either the same CE instrument or using another comparable CE instrument to measure fragment sizes.

21. The DNA analysis instrument of claim 18, wherein the DNA analysis instrument accesses the pre-computed model remotely.

22. The DNA analysis instrument of claim 18, further comprising a synthetic allelic ladder database storing a plurality of synthetic allelic ladders that is accessed by the fitter prior to dynamically generating the first synthetic allelic ladder using the pre-computed model, in order to determine if any stored synthetic allelic ladder fits the test fragment sizing data sufficiently for identifying which of the plurality of alleles are present in the biological sample.

23. The DNA analysis instrument of claim 22, wherein the DNA profile generator accesses the synthetic allelic ladder database remotely.

24. A method of testing a biological sample comprising deoxyribonucleic acid (DNA) molecules for presence of a plurality of alleles, wherein DNA fragments obtained using the biological sample and corresponding to different alleles of the plurality of alleles have different fragment sizes, the method comprising:
obtaining test fragment sizing data by migrating and scanning, using a capillary electrophoresis (CE) instrument, a plurality of fluorescently labelled DNA fragments corresponding to the biological sample;
using the test fragment sizing data to search a stored allelic ladder library, wherein the stored allelic ladder library comprises one or more stored synthetic allelic ladders that have been synthetically generated using a pre-computed model, the pre-computed model having been derived based on statistical analysis of a plurality of fragment sizing data sets obtained from a plurality of sample runs previously conducted on allelic ladder samples using either the same CE instrument or using another comparable CE instrument to measure fragment sizes;
determining whether the one or more stored allelic ladders fits the test fragment sizing data sufficiently to comprise a sufficiently fitting allelic ladder for identifying which of the plurality of alleles are present in the biological sample;
if the one or more stored allelic ladders does not fit the test fragment sizing data sufficiently, then dynamically generating one or more additional synthetic allelic ladders using the pre-computed model based on varying one or more parameters of the pre-computed model and determining whether any of the one or more additional synthetic allelic ladders fits the test fragment sizing data sufficiently to comprise a sufficiently fitting allelic ladder for identifying which of the plurality of alleles are present in the biological sample; and
once a sufficiently fitting allelic ladder is identified, using the sufficiently fitting allelic ladder to determine which of the plurality of alleles are present in the biological sample.

25. The method of claim 24, wherein the pre-computed model is based on principal component analysis (PCA).

26. The method of claim 25, wherein the principal component analysis comprises determining a first principal component having a first principal component range, and a second principal component having a second principal component range.

27. The method of claim 26, wherein the stored allelic ladder library comprises a plurality of synthetic allelic ladders that are associated with different first principal component values across the first principal component range, and different second principal component values across the second principal component range.

28. The method of claim 26, wherein the principal component analysis further comprises determining a representative allelic ladder comprising a plurality of alleles, each associated with a representative fragment size, wherein the representative allelic ladder is associated with a set of reference conditions.

29. The method of claim 28, wherein determining the representative allelic ladder further comprises:
running a plurality of experimental sample runs on allelic ladder samples under the set of reference conditions; and
calculating the average fragment size of each of the plurality of alleles in the experimental sample runs.

30. The method of claim 28, wherein determining the representative allelic ladder further comprises:
selecting a subset of the plurality of fragment sizing data sets that are within a specified range of the set of reference conditions; and
calculating the average fragment size of each of the plurality of alleles.

31. The method of claim 28, wherein the determining the representative allelic ladder further comprises: generating a preliminary migration model without determining a representative allelic ladder, wherein the preliminary migration model generates a representative synthetic allelic ladder corresponding to the set of reference conditions.

32. The method of claim 28, further comprising designating a fragment sizing data set of the plurality of fragment sizing data sets that is a sufficient fit to the representative synthetic allelic ladder as the representative allelic ladder.

33. The method of claim 28, further comprising:
finding a subset of the plurality of fragment sizing data sets, wherein each fragment sizing data set in the subset comprises a sufficient fit to the representative allelic ladder; and
calculating an average fragment size for each of the alleles in the subset.

34. The method of claim 28, further comprising linearly combining the first and second principal components to align with a temperature component and a gel degradation component, and setting a first reference condition at a center value of the temperature component, and setting a second reference condition at an upper value of the gel degradation component.

35. The method of claim 28, further comprising:
for each of the plurality of fragment sizing data sets, calculating a deviation value for each allele in the fragment sizing data set by subtracting the reference fragment size value from the data set fragment size value;
storing a matrix comprising the deviation values for the plurality of fragment sizing data sets; and
performing one or more principal component analysis matrix operations to determine principal components.

36. The method of claim 24, wherein the pre-computed model comprises an empirical model generated by:
defining a first variable and a second variable wherein the first variable and the second variable impact migration in the pre-computed model;
determining a first experimental range for the first variable and a second experimental range for the second variable;
selecting a reference condition within the first and second experimental ranges;
conducting a first series of calibration sample runs across the first experimental range for the first variable while holding the second variable constant at the reference condition, and a second series of calibration sample runs across the second experimental range for the second variable while holding the second variable constant at the reference condition;
defining a first parameter for the first variable and a second parameter for the second variable such that the first and second parameters are zero at the reference condition; and the first parameter comprises a non-zero value at a deviation of the first variable from the reference condition, and the second parameter comprises a non-zero value at a deviation of the second variable from the reference condition;
for the first and second variables, determining regression parameters and aggregating a slope of each allele in first and second plots to generate a first characteristic component and a second characteristic component; and
generating a reference ladder by aggregating the intercepts for the slopes of each of the alleles in the calibration sample.

37. The method of claim 24, wherein the stored allelic ladder library further comprises one or more stored native allelic ladders.

38. A non-transitory computer readable medium comprising a memory storing one or more instructions which, when executed by one or more processors of at least one computing device, perform validation of a DNA analysis instrument for testing a biological sample comprising one or more deoxyribonucleic acid (DNA) molecules, wherein the DNA molecule comprises one or more DNA loci and each DNA locus is associated with a plurality of alleles, by:
obtaining test fragment sizing data corresponding to fragment sizing values corresponding to a plurality of fragments of a control biological sample, the plurality of fragments detected by an electrophoresis genetic analyzer of the DNA analysis instrument; and
using a pre-computed model to dynamically generate one or more first synthetic allelic ladders, the pre-computed model having been derived based on statistical analysis of a plurality of fragment sizing data sets obtained from a plurality of sample runs previously conducted on allelic ladder biological samples using either the same electrophoresis instrument or using another comparable electrophoresis instrument to measure fragment sizes;
determining whether the first synthetic allelic ladder fits the control sample fragment sizing data sufficiently for identifying which of the plurality of alleles are present in the control biological sample and satisfies a pre-specified set of validation criteria;
if the first synthetic allelic ladder does not fit the control sample fragment sizing data sufficiently, then generating one or more additional synthetic allelic ladders based on varying one or more parameters of the pre-computed model and determining whether any of the one or more additional synthetic allelic ladders fits the test fragment sizing data sufficiently for identifying which of the plurality of alleles are present in the control biological sample and satisfies a pre-specified set of validation criteria; and
once a sufficiently fitting synthetic allelic ladder is identified, determining whether the plurality of alleles of the control biological sample match a corresponding plurality of alleles of the sufficiently fitting synthetic allelic ladder.

39. A non-transitory computer readable medium comprising a memory storing one or more instructions which, when executed by a one or more processors of at least one computing device, perform testing of a biological sample comprising one or more deoxyribonucleic acid (DNA) molecules, wherein the DNA molecule comprises one or more DNA loci and each DNA locus is associated with a plurality of alleles, by:
obtaining test fragment sizing data by migrating and scanning, using an analysis instrument, a plurality of labelled DNA fragments corresponding to the biological sample;
using a pre-computed model to dynamically generate at least one first synthetic allelic ladder, the pre-computed model based on analysis of a plurality of fragment sizing data sets obtained from a plurality of previously conducted sample runs using either the same analysis instrument or using another comparable analysis instrument to measure fragment sizes;
determining whether any of the first synthetic allelic ladders fit the test fragment sizing data sufficiently for identifying which of the plurality of alleles are present in the biological sample;
if first synthetic allelic ladder does not fit the test fragment sizing data sufficiently, then generating one or more additional synthetic allelic ladders based on varying one or more parameters of the pre-computed model and determining whether any of the one or more additional synthetic allelic ladders fits the test fragment sizing data sufficiently for identifying which of the plurality of alleles are present in the biological sample; and
once a sufficiently fitting synthetic allelic ladder is identified, using the sufficiently fitting synthetic allelic ladder to determine which of the plurality of alleles are present in the biological sample.

40. The non-transitory computer readable medium of claim 39, wherein the analysis instrument comprises a capillary electrophoresis (CE) instrument.

41. The non-transitory computer readable medium of claim 39, wherein the plurality of previously conducted sample runs comprises one or more allelic ladder sample runs.

42. The non-transitory computer readable medium of claim 39, wherein the plurality of previously conducted sample runs comprises one or more one or more test sample runs from other biological samples.

43. The non-transitory computer readable medium of claim 39, wherein the one or more additional synthetic allelic ladders are generated after a sufficiently fitting allelic ladder is identified, in order to satisfy one or more optimization criteria.
